(19) United States
(12) Patent Application Publication
(10) Pub. No.: US 2012/0094874 A1
(43) Pub. Date: Apr. 19, 2012

(54) STABILIZED IMMUNOGLOBULIN CONSTANT DOMAINS

(75) Inventors: Florian Rüker, Vienna (AT); Gordana Wozniak-Knopp, Vienna (AT); Gottfried Himmler, Gross Enzersdorf (AT)

(73) Assignee: F-STAR BIOTECHNOLOGISCHE FORSCHUNGS-UND ENTWICKLUNGSGES. M.B.H, Vienna (AT)

(21) Appl. No.: 13/377,817

(22) PCT Filed: Jul. 1, 2010
(86) PCT No.: PCT/EP2010/059408

§ 371 Date: Dec. 12, 2011

(30) Foreign Application Priority Data
Jul. 9, 2009 (EP) 09165029.1

(51) Int. Cl. C40B 40/10; C12P 21/00; C07K 16/00

(52) U.S. Cl. 506/18; 530/387.3; 435/69.6

(57) ABSTRACT

The invention refers to a multidomain modular antibody comprising at least one constant antibody domain, which is mutated to form an artificial disulfide bridge by introducing at least one Cys residue into the amino acid sequence through mutagenesis of said constant domain to obtain an intra-domain or inter-domain disulfide bridge within the framework region, libraries based on such antibodies and methods of producing.

The invention refers to a multidomain immunoglobulin comprising at least one constant antibody domain, which is stabilized.

Monoclonal antibodies have been widely used as therapeutic binding agents. The basic antibody structure will be explained here using as an example an intact IgG1 immunoglobulin.

Two identical heavy (H) and two identical light (L) chains combine to form the Y-shaped antibody molecule. The heavy chains each have four domains. The amino terminal variable domains (VH) are at the tips of the Y. In the case of IgG, IgD and IgA, these are followed by three constant domains: CH1, CH2, and the carboxy-terminal CH3, at the base of the Y's stem. In the case of IgM and IgE there are four different constant domains. A short stretch, the switch, connects the heavy chain variable and constant regions. The hinge connects CH2 and CH3 (the Fc fragment) to the remainder of the antibody (the Fab fragments). One Fc and two identical Fab fragments can be produced by proteolytic cleavage of the hinge in an intact antibody molecule. The light chains are constructed of two domains, variable (VL) and constant (CL), separated by a switch.

Disulfide bonds in the hinge region connect the two heavy chains. The light chains are coupled to the heavy chains by additional disulfide bonds. Asn-linked carbohydrate moieties are attached at different positions in constant domains depending on the class of immunoglobulin. For human IgG1 two disulfide bonds in the hinge region, between Cys226 and Cys229 pairs, unite the two heavy chains. The light chains are coupled to the heavy chains by two additional disulfide bonds, between the Cys following Ser221 in the CH1 domain and Cys214s in the CL domain. Carbohydrate moieties are attached to Asn297 of each CH2, generating a pronounced bulge in the stem of the Y. The numbers here are given according to the Kabat numbering scheme.

These features have profound functional consequences. The variable regions of both the heavy and light chains (VH) and (VL) lie at the “tips” of the Y, where they are positioned to react with antigen. This tip of the molecule is the side on which the N-terminus of the amino acid sequence is located. The stem of the Y projects in a way to efficiently mediate effector functions such as the activation of complement and interaction with Fc receptors, or ADCC and ADCP. Its CH2 and CH3 domains bulge to facilitate interaction with effector proteins. The C-terminus of the amino acid sequence is located on the opposite side of the tip, which can be termed “bottom” of the Y.

Two types of light chain, termed lambda (λ) and kappa (κ), are found in antibodies. A given immunoglobulin either has kappa chains or lambda chains, never one of each. No functional difference has been found between antibodies having lambda or kappa light chains.

Each domain in an antibody molecule has a similar structure of two beta sheets packed tightly against each other in a compressed antiparallel beta barrel. This conserved structure is termed the immunoglobulin fold. The immunoglobulin fold of constant domains contains a 3-stranded sheet packed against a 4-stranded sheet. The fold is stabilized by hydrogen bonding between the beta strands of each sheet, by hydrophobic bonding between residues of opposite sheets in the interior, and by a disulfide bond between the sheets. The 3-stranded sheet comprises strands C, F, and G, and the 4-stranded sheet has strands A, B, E, and D. The letters A through G denote the sequential positions of the beta strands along the amino acid sequence of the immunoglobulin fold.

The fold of variable domains has 9 beta strands arranged in two sheets of 4 and 5 strands. The 5-stranded sheet is structurally homologous to the 3-stranded sheet of constant domains, but contains the extra strands C′ and C″. The remainder of the strands (A, B, C, D, E, F, G) have the same topology and similar structure as their counterparts in constant domain immunoglobulin folds. A disulfide bond links strands B and F in opposite sheets, as in constant domains.

The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are loops that connect beta strands B—C, C′—C″, and F-G of the immunoglobulin fold. The residues in the CDRs vary from one immunoglobulin molecule to the next, imparting antigen specificity to each antibody.

The VL and VH domains at the tips of antibody molecules are closely packed such that the 6 CDRs (3 on each domain) cooperate in constructing a surface (or cavity) for antigen-specific binding. The natural antigen binding site of an antibody thus is composed of the loops which connect strands B—C, C′—C″, and F-G of the light chain variable domain and strands B—C, C′—C″, and F-G of the heavy chain variable domain.

The loops, which are not CDR-loops in a native immunoglobulin, apart from the antigen-binding pocket, which is determined by the CDR loops and optionally adjacent loops within the CDR loop region that contribute to the antigen-binding pocket, do not have antigen binding or epitope binding specificity, but contribute to the correct folding of the entire immunoglobulin molecule are therefore called structural loops for the purpose of this invention.

Prior art documents show that the immunoglobulin scaffold has been employed so far for the purpose of manipulating the existing antigen binding site, thereby introducing novel binding properties. In most cases the CDR regions have been engineered for various antigen binding, in other words, in the case of the immunoglobulin fold, only the natural antigen binding site has been modified in order to change its binding affinity or specificity. A vast body of literature exists which describes different formats of such manipulated immunoglobulins, frequently expressed in the form of single-chain Fv fragments (scFv) or Fab fragments, either displayed on the surface of phage particles or solubly expressed in various prokaryotic or eukaryotic expression systems. Various immunoglobulin libraries have been proposed in the art to obtain specific immunoglobulin binders. However, the scaffolds used for preparing such libraries were limited, because of possible deterioration of the framework when engineering the antigen-binding pocket.

The prior art also refers to stabilizing single CH2 antibody domains. Gong et al (J. Biol. Chem. (2009) 284 (21): 14203-14210) describe isolated, unglycosylated human CH2 single domains, which are relatively unstable to thermally induced unfolding. A mutant CH2 domain was engineered, which had an additional disulfide bond within the region of the native disulfide bond, i.e. between the N-terminal strand A and the C-terminal one G. Thereby a thermal stability with a Tm of up to 73° C. was obtained with the monomeric CH2. The engineered single domain CH2 domains, also called nanoantibodies, can be used as scaffolds (Dimitrov (2009) mAbs1:1, 26-28).

The dimerization of the CH3 domain is described to play a pivotal role in the assembly of an antibody. Mcauley et al (Protein Science (2008) 17:95-106) teach that the disulfide bond within the CH3 domain between Cys367 and Cys425 (according to the Kabat numbering scheme) is buried and highly conserved. This disulfide bond is not required for dimerization, since a recombinant human CH3 domain, even in the reduced state, existed as a dimer.

WO06072620A1 describes a method of engineering an immunoglobulin, which comprises a modification in a structural loop region to obtain new antigen binding sites. This method is broadly applicable to immunoglobulins and may be used to produce a library of immunoglobulins targeting a variety of antigens. A CH3 library has been shown to be useful for selecting specific binders to an antigen.

WO2009/000006A1 describes method of producing oligomers of antibody domains binding to a target and to a scaffold ligand.

WO2006/036834A1 describes biologically active peptides incorporated into an Fc domain.

There is a need to provide stable immunoglobulins for preparing respective libraries. It is thus the object of the invention to provide an improved immunoglobulin as a scaffold for antibody engineering.

The object is solved by the subject matter as claimed.

### SUMMARY OF THE INVENTION

According to the invention there is provided a multidomain modular antibody comprising at least one constant antibody domain, which is mutated to form an artificial disulfide bridge by introducing at least one Cys residue into the amino acid sequence through mutagenesis of said constant domain to obtain an intra-domain or inter-domain disulfide bridge within the framework region.

Preferably the modular antibody according to the invention comprises at least two constant domains connected by said artificial disulfide bridge.

The preferred modular antibody according to the invention has an antigen-binding region, preferably besides the site of mutation. Thus, the preferred modular antibody according to the invention has said at least one Cys residue introduced aside from an antigen binding site of the antibody.

The modular antibody according to the invention preferably is a full-length antibody or part of an antibody, such as an Fab, Fc or other combinations of at least one constant domain with at least one of a constant domain or a variable domain.

The modular antibody according to the invention preferably comprises the artificial disulfide bridge formed by introducing at least one Cys residue, wherein a single Cys residue would preferably be engineered to obtain an inter-domain bridge, such as between homodimeric domains. Two additional Cys residues within a domain would preferably be engineered to obtain an additional intra-domain disulfide bridge.

A preferred modular antibody according to the invention comprises a constant domain contributing to the antigen-binding function of the modular antibody, such as a constant domain which forms at least part of an antigen binding site.

According to a preferred embodiment the modular antibody according to the invention is used to provide for a novel scaffold for producing a modular antibody library.

According to the invention there is further provided a library of modular antibodies, which are mutagenized to obtain a randomized amino acid sequence within a loop region.

According to the invention there is further provided a method of producing a modular antibody according to the invention, which comprises the steps of
providing an modular antibody comprising at least two antibody domains, wherein at least one of the antibody domains is a constant domain,
mutating said constant domain to introduce a Cys residue within the framework region of said domain, and
expressing said modular antibody at oxidizing conditions to form a new disulfide bridge within the molecule.

According to the preferred method at least two constant domains are mutated to introduce a Cys residue. In an equivalent embodiment any other artificial or alternative thiol forming amino acid or amino acid analogue may be engineered into the amino acid sequence to form the artificial disulfide bridge. The amino acid sequence is preferably mutated by insertion, or substitution.

In a preferred method according to the invention the Cys residue is introduced aside from an antigen binding site of the antibody. Thus, the biological activity or antigen-binding property would not be hindered by such Cys engineering or disulfide bond formation.

The further preferred method according to the invention provides for the mutation of said constant domain at a position within the framework region of said domain, e.g. within the structural loop region or the beta-sheet region, such as selected from the group consisting of following amino acid positions:

Sheet A: 1-15.1

Sheet B: 16-26

Sheet C: 39-45.1

Sheet D: 77-84

Sheet E: 85.1-96

Sheet F: 96.2-104

Sheet G: 118-129

Numbers are according to the IMGT numbering scheme.

Preferred sites of introducing appropriate artificial disulfide bridges are shown in Table 1. Though the numbering refers to human IgG1 antibody domains, the analogous positions of other antibody domains, e.g. of different antibody class or different origin, like a mammalian species other than human, or a mutant or variant antibody domain, may be chosen for this purpose of engineering an artificial disulfide bridge.

TABLE 1
Preferred sites of bridge piers of an artificial
disulfide bridge within a constant immunoglobulin
domain, particularly IgG1 of human origin.

| Residue No | Residue No |
|---|---|
| According to IMGT | According to IMGT |
| 1 | 110 |
| 2 | 25 |
| 2 | 27 |
| 2 | 28 |
| 1.1 | 29 |
| 3 | 26 |
| 1.2 | 110 |
| 4 | 119 |
| 5 | 24 |
| 1.5 | 85.4 |
| 6 | 119 |
| 6 | 121 |
| 7 | 22 |
| 9 | 13 |
| 9 | 19 |
| 9 | 21 |
| 9 | 123 |
| 10 | 12 |
| 10 | 13 |
| 11 | 34 |
| 11 | 36 |
| 12 | 36 |
| 13 | 17 |
| 13 | 19 |
| 14 | 19 |
| 15 | 115 |
| 15.1 | 16 |
| 15.1 | 17 |
| 19 | 96 |
| 21 | 89 |
| 23 | 87 |
| 23 | 104 |
| 25 | 85 |
| 26 | 27 |
| 26 | 85.1 |
| 27 | 85.3 |
| 28 | 85.2 |
| 29 | 32 |
| 32 | 109 |
| 33 | 32 |
| 33 | 83 |
| 33 | 85.2 |
| 36 | 107 |
| 40 | 105 |
| 41 | 45.1 |
| 41 | 45.3 |
| 42 | 45.1 |
| 42 | 103 |
| 78 | 89 |
| 80 | 87 |
| 81 | 86 |
| 83 | 85 |
| 83 | 85.1 |
| 83 | 85.2 |
| 84 | 85.1 |
| 84.2 | 85.3 |
| 84.4 | 85.3 |
| 91 | 95 |
| 92 | 95 |
| 95 | 100 |
| 101 | 122 |
| 102 | 121 |
| 103 | 120 |
| 105 | 118 |
| 106 | 117 |
| 107 | 116 |
| 108 | 112 |
| 108 | 113 |
| 108 | 115 |
| 112 | 115 |
| 113 | 115 |
| 122 | 125 |
| 124 | 30 |

A preferred method according to the invention provides for mutating a constant domain, which contributes to antigen-binding, such as a constant domain which forms at least part of an antigen binding site.

In a preferred method according to the invention the modular antibody is expressed by a host cell at disulfide forming conditions, e.g. expressed and/or secreted to form disulfide bonds, such as by expression in the periplasmic space of E. coli or by expression as a secreted protein in a eukaryotic expression system such as yeast or mammalian cells.

The invention further provides for a method of introducing a disulfide bond into the framework of a constant domain to increase thermostability of a multidomain modular antibody.

According to a further embodiment of the invention, there is provided a method of introducing a disulfide bond into the framework of a constant domain to improve antigen-binding of a multidomain modular antibody.

### FIGURES

FIG. 1 shows the sequence of the mutant Fc. The mutated residues in which this sequence differs from that of wildtype Fc are underlined.

FIG. 2 shows the sequence of a wild-type Fc with mutations to introduce Cys residues (mutated Cysteines are underlined). FIG. 2 a. shows the sequence of Fc CysP2; FIG. 2 b. shows the sequence of Fc CysP4, as described in Example 2.

FIG. 3 shows the sequence of a wild-type Fc with mutations to introduce Cys residues (mutated Cysteines are underlined). FIG. 3 a. shows the sequence of Fc CysP24; FIG. 3 b. shows the sequence of Fc CysP2Cys, as described in Example 2.

FIG. 4 shows the sequence of a Her2/neu binding Fc with mutations to introduce Cys residues (mutated Cysteines are underlined). FIG. 4 a. shows the sequence of Fc H10-03-6 without a Cys mutation; FIG. 4 b. shows the sequence of Fc H10-03-6Cys; FIG. 4 c. shows the sequence of Fc H10-03-6CysP2; FIG. 4 d. shows the sequence of Fc H10-03-6CysP2Cys, as described in Example 3.

### DETAILED DESCRIPTION OF THE INVENTION

### Definitions

Specific terms as used throughout the specification have the following meaning.

The term “antigen” or “target” as used according to the present invention shall in particular include all antigens and target molecules capable of being recognised by a binding site of a modular antibody. Specifically preferred antigens as targeted by the modular antibody according to the invention are those antigens or molecules, which have already been proven to be or are capable of being immunologically or therapeutically relevant, especially those, for which a clinical efficacy has been tested.

The term specifically comprises molecules selected from the group consisting of allergens, tumor associated antigens, self antigens including cell surface receptors, enzymes, Fc-receptors, FcRn, HSA, IgG, interleukins or cytokines, proteins of the complement system, transport proteins, serum molecules, bacterial antigens, fungal antigens, protozoan antigen and viral antigens, also molecules responsible for transmissible spongiform encephalitis (TSE), such as prions, infective or not, and markers or molecules that relate to inflammatory conditions, such as pro-inflammatory factors, multiple sclerosis or Alzheimer's disease, or else haptens.

The antigen is either recognized as a whole target molecule or as a fragment of such molecule, especially substructures of targets, generally referred to as epitopes (e.g. B-cell epitopes, T-cell epitopes), Epitopes are understood to be immunologically relevant, i.e. are recognisable by natural or monoclonal antibodies. Therefore, the term “epitope” as used herein according to the present invention shall mean a molecular structure which may completely make up a specific binding partner or be part of a specific binding partner to a binding site of modular antibody of the present invention. The term epitope may also refer to haptens. Chemically, an epitope may either be composed of a carbohydrate, a peptide, a fatty acid, an organic, biochemical or inorganic substance or derivatives thereof and any combinations thereof. If an epitope is a polypeptide, it will usually include at least 3 amino acids, preferably 8 to 50 amino acids, and more preferably between about 10 to 20 amino acids in the peptide. There is no critical upper limit to the length of the peptide, which could comprise nearly the full length of a polypeptide sequence of a protein. Epitopes can be either linear or conformational epitopes. A linear epitope is comprised of a single segment of a primary sequence of a polypeptide chain. Linear epitopes can be contiguous or overlapping. Conformational epitopes are comprised of amino acids brought together by folding of the polypeptide to form a tertiary structure and the amino acids of the epitope are not necessarily adjacent to one another in the linear sequence. Specifically, epitopes are at least part of diagnostically relevant molecules, i.e. the absence or presence of an epitope in a sample is qualitatively or quantitatively correlated to either a disease or to the health status of a patient or to a process status in manufacturing or to environmental and food status. Epitopes may also be at least part of therapeutically relevant molecules, i.e. molecules which can be targeted by the specific binding domain which changes the course of the disease.

“Artificial” with reference to a disulfide bridge (“S—S bridge”) means that the S—S bridge is not naturally formed by the wild-type modular antibody, but is formed by an engineered mutant of a parent molecule, wherein at least one foreign amino acid contributes to the disulfide bonding. The site-directed engineering of artificial disulfide bridges clearly differentiates from those naturally available in native immunoglobulins or in modular antibodies, such as those described in WO2009/000006A1, because at least one of the sites of bridge piers of an artificial disulfide bridge is typically located aside from the positions of Cys residues in the wild-type antibody, thus, providing for an alternative or additional disulfide bridge within the framework region.

The term “expression system” refers to nucleic acid molecules containing a desired coding sequence and control sequences in operable linkage, so that hosts transformed or transfected with these sequences are capable of producing the encoded proteins. In order to effect transformation, the expression system may be included on a vector; however, the relevant DNA may then also be integrated into the host chromosome. Alternatively, an expression system can be used for in vitro transcription/translation.

The term “foreign” in the context of amino acids shall mean a newly introduced amino acid in an amino acid sequence, which is usually naturally occurring, but foreign to the site of modification, or a substitute of a naturally occurring amino acid.

The term “framework” or “framework region” shall refer to those conserved regions of a modular antibody that are located outside the CDR loop region of an antibody domain including the structural loop regions. The framework region usually comprises or consists of a beta-sheet region of an immunoglobulin domain. Typically, the Cys mutations according to the invention would be in a framework region, where they do not sterically hinder any antigen-binding site of a modular antibody. Thus, it is understood that the framework region of a modular antibody according to the invention typically is aside from antigen-binding sequences. Any incorporation of biologically active peptide sequences into the loop region of an Fc domain according to WO2006/036834A1 is considered a potential binding site, where disulfide bridges within the peptide sequences would be avoided to maintain the biological activity of the peptide sequence.

The term “immunoglobulin” as used according to the present invention is defined as polypeptides or proteins that may exhibit mono- or bi- or multi-specific, or mono-, bi- or multivalent binding properties, preferably at least two, more preferred at least three specific binding sites for epitopes of e.g. antigens, effector molecules or proteins either of pathogen origin or of human structure, like self-antigens including cell-associated or serum proteins. The term immunoglobulin as used according to the invention also includes functional fragments of an antibody, such as Fc, Fab, scFv, single chains of pairs of immunoglobulin domains, like single chain dimers of CH1/CL domains, Fv, or dimers such as VH/VL, CH1/CL, CH2/CH2, CH3/CH3, or other derivatives or combinations of the immunoglobulins. The definition further includes domains of the heavy and light chains of the variable region (such as dAb, Fd, Vl, Vk, Vh, VHH) and the constant region or individual domains of an intact antibody such as CH1, CH2, CH3, CH4, Cl and Ck, as well as mini-domains consisting of at least two beta-strands of an immunoglobulin domain connected by a structural loop, or recombined antibody domains, such as strand-exchange engineered domains (SEEDbodies), like those interdigitating beta-strand segments of human IgG and IgA CH3 domains.

The term “immunoglobulin-like molecule” as used according to the invention refers to any antigen-binding protein, in particular to a human protein, which has a domain structure that can be built in a modular way. Immunoglobulin-like molecules as preferably used for the present invention are T-cell receptors (TCR), fibronectin, transferrin, CTLA-4, single-chain antigen receptors, e.g. those related to T-cell receptors and antibodies, antibody mimetics, adnectins, anticalins, phylomers, repeat proteins such as ankyrin repeats, avimers, Versabodies, scorpio toxin based molecules, and other non-antibody protein scaffolds with antigen binding properties.

Ankyrin repeat (AR), armadillo repeat (ARM), leucine-rich repeat (LRR) and tetratricopeptide repeat (TPR) proteins are the most prominent members of the protein class of repeat proteins. Repeat proteins are composed of homologous structural units (repeats) that stack to form elongated domains. The binding interaction is usually mediated by several adjacent repeats, leading to large target interaction surfaces.

Avimers contain A-domains as strings of multiple domains in several cell-surface receptors. Domains of this family bind naturally over 100 different known targets, including small molecules, proteins and viruses. Truncation analysis has shown that a target is typically contacted by multiple A-domains with each domain binding independently to a unique epitope. The avidity generated by combining multiple binding domains is a powerful approach to increase affinity and specificity, which these receptors have exploited during evolution.

Anticalins are engineered human proteins derived from the lipocalin scaffold with prescribed binding properties typical for humanized antibodies. Lipocalins comprise 160-180 amino acids and form conical beta-barrel proteins with a ligand-binding pocket surrounded by four loops. Small hydrophobic compounds are the natural ligands of lipocalins, and different lipocalin variants with new compound specificities (also termed ‘anticalins’) could be isolated after randomizing residues in this binding pocket.

Single chain antigen receptors contain a single variable domain and are 20% smaller than camelid single domain antibodies.

Phylomers are peptides derived from biodiverse natural protein fragments.

It is understood that the term “modular antibody”, “immunoglobulin”, “immunoglobulin-like proteins” includes a derivative thereof as well. A derivative is any combination with one or more modular antibodies of the invention and or a fusion protein in which any domain or minidomain of the modular antibody of the invention may be fused at any position of one or more other proteins (such as other modular antibodies, immunoglobulins, ligands, scaffold proteins, enzymes, toxins and the like). A derivative of the modular antibody of the invention may also be obtained by association or binding to other substances by various chemical techniques such as covalent coupling, electrostatic interaction, disulphide bonding etc. The other substances bound to the immunoglobulins may be lipids, carbohydrates, nucleic acids, organic and inorganic molecules or any combination thereof (e.g. PEG, prodrugs or drugs). A derivative would also comprise an antibody with the homologous amino acid sequence, which may contain non-natural or chemically modified amino acids. Further derivatives of modular antibodies are provided as fragments thereof, containing at least a framework region and a loop region.

“Modular antibodies” as used according to the invention are defined as antigen-binding molecules, like human antibodies, composed of at least one polypeptide module or protein domain, preferably in the natural form. The term “modular antibodies” includes antigen-binding molecules that are either immunoglobulins, immunoglobulin-like proteins, or other proteins exhibiting modular formats and antigen-binding properties similar to immunoglobulins or antibodies, which can be used as antigen-binding scaffolds, preferably based on human proteins.

The term “multidomain modular antibody” as used according to the invention refers to a modular antibody comprising at least two modular antibodies and domains, respectively.

As used herein, the term “specifically binds” or “specific binding” refers to a binding reaction which is determinative of the cognate ligand of interest in a heterogeneous population of molecules. Thus, under designated conditions (e.g. immunoassay conditions), the modular antibody binds to its particular target and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10 fold different, preferably the difference is at least 100 fold, and more preferred a least 1000 fold.

“Scaffold” shall mean a temporary framework either natural or artificial used to support the molecular structure of a polypeptide in the construction of variants or a repertoire of the polypeptide. It is usually a modular system of polypeptide domains that maintains the tertiary structure or the function of the parent molecule. Exemplary scaffolds are modular antibodies, which may be mutagenized to produce variants within said scaffold, to obtain a library.

A “structural loop” or “non-CDR-loop” according to the present invention is to be understood in the following manner: modular antibodies, immunoglobulins or immunoglobulin-like substances are made of domains with a so called immunoglobulin fold. In essence, antiparallel beta sheets are connected by loops to form a compressed antiparallel beta barrel. Loop regions of constant domains or loop regions of variable domains that are apart from the CDR loop region, i.e. non-CDR loops, are called structural loops. In the variable region, some of the loops of the domains contribute essentially to the specificity of the antibody, i.e. the binding to an antigen by the natural binding site of an antibody. These loops are called CDR-loops. The CDR loops are located within the CDR loop region, which may in some cases also include the variable framework region (called “VFR”) adjacent to the CDR loops. It is known that VFRs may contribute to the antigen binding pocket of an antibody, which generally is mainly determined by the CDR loops. Thus, those VFRs are considered as part of the CDR loop region, and would not be appropriately used for engineering new antigen binding sites. Contrary to those VFRs within the CDR loop region or located proximal to the CDR loops, other VFRs of variable domains would be particularly suitable for engineering an additional antigen binding site. Those are the structural loops of the VFRs located opposite to the CDR loop region, or at the C-terminal side of a variable immunoglobulin domain.

The term “variable binding region” sometimes called “CDR region” as used herein refers to molecules with varying structures capable of binding interactions with antigens. Those molecules can be used as such or integrated within a larger protein, thus forming a specific region of such protein with binding function. The varying structures can be derived from natural repertoires of binding proteins such as immunoglobulins or phylomers or synthetic diversity, including repeat-proteins, avimers and anticalins. The varying structures can as well be produced by randomization techniques, in particular those described herein. These include mutagenized CDR or non-CDR regions, loop regions of immunoglobulin variable domains or constant domains.

Modified binding agents with different modifications at specific sites are referred to as “variants”. Variants of a scaffold are preferably grouped to form libraries of binding agents, which can be used for selecting members of the library with predetermined functions. In accordance therewith, a loop region of a binding agent comprising positions within one or more loops potentially contributing to a binding site, is preferably mutated or modified to produce libraries, preferably by random, semi-random or, in particular, by site-directed random mutagenesis methods, in particular to delete, exchange or introduce randomly generated inserts into loops, preferably into structural loops. Alternatively preferred is the use of combinatorial approaches. Any of the known mutagenesis methods may be employed, among them cassette mutagenesis. These methods may be used to make amino acid modifications at desired positions of the modular antibody of the present invention. In some cases positions are chosen randomly, e.g. with either any of the possible amino acids or a selection of preferred amino acids to randomize loop sequences, or amino acid changes are made using simplistic rules. For example all residues may be mutated preferably to specific amino acids, such as alanine, referred to as amino acid or alanine scanning. Such methods may be coupled with more sophisticated engineering approaches that employ selection methods to screen higher levels of sequence diversity.

All numbering of the amino acid sequences of the modular antibody according to the invention is according to the IMGT numbering scheme (IMGT, the international ImMunoGeneTics, Lefranc et al., 1999, Nucleic Acids Res. 27: 209-212).

Therefore, the multidomain modular antibody according to the invention comprises at least one constant antibody domain, and is mutated to form an artificial disulfide bridge within the framework region. It was surprising that such a modular antibody could have a significantly increased thermostability. The multidomain structure of the modular antibody according to the invention is sometimes called “multimeric”.

In the multidomain format the modular antibody according to the invention is preferably composed of at least two domains, more preferred at least 3, 4, 5, 6, 7, 8, 9 up to 10 domains, in particular antibody domains, such as to obtain full length antibodies or fragments of antibodies containing at least one constant domains combined with at least one further constant and/or at least one variable domain.

The preferred size is at least 20 kD. Modular antibody single domains usually have a molecular size of 10-15 kD, thus a molecule based on 2 modular antibody domains would have a molecular size of 20-30 kD, depending on the glycosylation or any additional conjugation of pharmacologically active substances, like toxins or peptides.

The preferred format is an oligomer composed of modular antibody domains, preferably 2 to 4 domains, with or without a covalent bond or a hinge region. Formats based on the combination of at least one pair of modular antibody domains are particularly preferred.

It is feasible to provide the preferred modular antibody of the invention as a pair of single domain antibodies. Antibody domains tend to dimerize upon expression, either as a homodimer, like an Fc, or a heterodimer, like an Fab. The dimeric structure is thus considered as a basis for the preferred stable molecule. The preferred dimers of immunoglobulin domains are selected from the group consisting of single domain dimers, like VH/VL, CH₁/CL (kappa or lambda) and CH3/CH3. Since CH2 single domains would not dimerize as such, a pair of CH2 domains would only be preferred, if an interchain disulfide bridge would be engineered into the molecule. A pair of single, monomeric CH2 domains, which are not dimerized, would not be preferably used. Dimers or oligomers of modular antibody domains according to the invention can also be provided as single chain or two chain molecules, in particular those linking the C-terminus of one domain to the N-terminus of another.

If more than one domain is present in the modular antibody these domains may be of the same type or of varying types (e.g. CH1-CH1-CH2, CH3-CH3, (CH2)2-(CH3)2, with or without the hinge region). Of course also the order of the single domains may be of any kind (e.g. CH1-CH3-CH2, CH4-CH1-CH3-CH2).

The invention preferably refers to part of antibodies, such as IgG, IgA, IgM, IgD, IgE and the like. The modular antibodies of the invention may also be a functional antibody fragment such as Fab, Fab2, scFv, Fv, Fc, Fcab™ (registered trademark of f-star Biotechnologische Forschungs- and Entwicklungsges.m.b.H.), an antigen-binding Fc, or parts thereof, or other derivatives or combinations of the immunoglobulins such as minibodies, domains of the heavy and light chains of the variable region (such as dAb, Fd, VL, including Vlambda and Vkappa, VH, VHH) as well as mini-domains consisting of two beta-strands of an immunoglobulin domain connected by at least two structural loops, as isolated domains or in the context of naturally associated molecules. A particular embodiment of the present invention refers to the Fc fragment of an antibody molecule, either as antigen-binding Fc fragment (Fcab™) through modifications of the amino acid sequence or as conjugates or fusions to receptors, peptides or other antigen-binding modules, such as scFv.

A modular antibody according to the invention preferably comprises a heavy and/or light chain or a part thereof. A modular antibody according to the invention may comprise a heavy and/or light chain, at least one variable and/or constant domain, or a part thereof including a minidomain.

A constant domain is an immunoglobulin fold unit of the constant part of an immunoglobulin molecule, also referred to as a domain of the constant region (e.g. CH1, CH2, CH3, CH4, Ckappa, Clambda).

A variable domain is an immunoglobulin fold unit of the variable part of an immunoglobulin, also referred to as a domain of the variable region (e.g. Vh, Vkappa, Vlambda, Vd).

An exemplary modular antibody according to the invention comprises a constant domain selected from the group consisting of CH1, CH2, CH3, CH4, Igkappa-C, Iglambda-C, combinations, derivatives or a part thereof including a mini-domain, with at least one framework or loop region, and is characterised in that said at least one framework region comprises at least one amino acid modification forming at least one artificial disulfide bridge besides a loop region, which may be part of or comprise a binding site. Preferably the framework is mutated for disulfide bond formation in such a way, that a binding site could be engineered within the loop region or, if already present, the binding site, represented by either a CDR loop region or a structural region, would be essentially maintained, e.g. with a loss of affinity (Kd) in binding an antigen, which is not more than 10⁻² M, preferably not more than 10⁻¹ M.

Another modular antibody according to the invention can consist of a variable domain of a heavy or light chain, combinations, derivatives or a part thereof including a minidomain, with at least one framework region, and is characterised in that said at least one framework region comprises at least one amino acid modification forming at least one additional disulfide bond.

The artificial disulfide bridge of the present invention may be engineered within an antibody domain (“intradomain bridge”), which would stabilize the beta-sheet structure or bridging the domains (“interdomain bridge”) or chains of domains (“interchain bridge”), to constrain the structure of the multimeric modular antibody according to the invention and support its interaction with potential binding partners.

The artificial disulfide bridge as engineered according to the invention is provided as a covalent bond, usually derived by the coupling of two thiol groups. The linkage is also called an SS-bond or a persulfide. The disulfide bond within molecules usually is about 2 angstrom in length. Thus, it was surprising that an artificial disulfide bond within the framework of a modular antibody according to the invention could stabilize the molecule without destroying its framework.

Disulfides where the two amino acid groups are the same are called symmetric, examples being diphenyl disulfide and dimethyl disulfide. When the two R groups are not identical, the compound is said to be an unsymmetric or mixed disulfide.

Disulfide bonds according to the invention are usually formed from the oxidation of sulfhydryl (—SH) groups, especially in biological contexts.

The preferred framework point mutations provide for newly introduced Cys residues into the amino acid sequence to form symmetric disulfide bridges upon oxidation, e.g. an interdomain bridge to form a dimer. Asymmetric bridges typically are intradomain or intrachain bridges. Oxidation of the respective thiol groups is achieved either through the recombinant protein expression or cultivation under oxidizing conditions, e.g. through expression by E. coli in the periplasmatic space, or upon secretion by a eukaryotic cell. Whereas reducing conditions within the cytoplasm would block the S—S bridging, oxidizing conditions within or outside the host cell would induce disulfide bonding. In vitro disulfide bonding is achieved by eventual reducing S—S bonds using a reducing agent, such as beta-mercaptoethanol, and folding or refolding by removing reducing agents, such as through dialysis or appropriate dilution. Standard methods for disulfide bonding are described by Bulaj G. (Biotechnol Adv. 2005 January; 23(1):87-92).

A variety of oxidants promote this reaction including air and hydrogen peroxide. Such reactions are thought to proceed via sulfenic acid intermediates. In the laboratory, iodine in the presence of base is commonly employed to oxidize thiols to disulfides. Several metals, such as copper(II) and iron(III) complexes effect this reaction. Alternatively, disulfide bonds in proteins often formed by thiol-disulfide exchange. Such reactions are mediated by enzymes in some cases and in other cases are under equilibrium control, especially in the presence of catalytic amount of base. Many specialized methods have been developed for forming disulfides, usually for applications in organic synthesis. Alternative amino acids, e.g. D-Cys instead of the natural L-Cys, are feasible.

The invention also provides a method of producing a modular antibody according to the invention, which employs the step of mutagenesis to introduce a Cys residue within the amino acid sequence. Mutations can be introduced by a variety of standard site directed mutagenesis methods.

For selecting the residues to introduce disulfide binds in frameworks, software programs can be used which predict at which positions newly introduced Cystein residues could lead to the formation of disulfide bridges. These software programs analyze crystal structures of proteins and measure e.g. the distance between C-beta atoms between pairs of residues. Those positions are preferably mutated, where the distance between two C-beta atoms is between about 3.4 and 42 angstrom.

Preferable sites for mutagenesis are as shown in Tables 2 and 3. Possible disulfide bridges that can be created by mutating the given pairs of residues can be read from the tables. Though the numbering refers to human IgG1 antibody domains, the analogous positions of other antibody domains, e.g. of different antibody class or different origin, like a mammalian species other than human, or a mutant or variant antibody domain, may be chosen for this purpose of engineering an artificial disulfide bridge.

TABLE 2
Preferred sites of bridge piers of an artificial disulfide bridge within a
constant immunoglobulin domain of an Fab region,
particularly of human origin

| | | | Residue | | | Residue |
|---|---|---|---|---|---|---|
| Antibody | | | No | | | No |
| domain | Residue | Chain | IMGT | Residue | Chain | IMGT |
| CL | | | | | | |
| Model | ARG | L | 1.5 | SER | L | 85.4 |
| Model | ALA | L | 1.1 | TYR | L | 29 |
| Model | PRO | L | 2 | LEU | L | 25 |
| Model | PRO | L | 2 | PHE | L | 28 |
| Model | SER | L | 3 | ASN | L | 26 |
| Model | PHE | L | 5 | LEU | L | 24 |
| Model | ILE | L | 6 | LYS | L | 119 |
| Model | PHE | L | 7 | VAL | L | 22 |
| Model | PRO | L | 9 | ALA | L | 19 |
| Model | SER | L | 10 | GLU | L | 12 |
| Model | SER | L | 10 | GLN | L | 13 |
| Model | ALA | L | 19 | TYR | L | 96 |
| Model | VAL | L | 21 | LEU | L | 89 |
| Model | CYS | L | 23 | SER | L | 87 |
| Native | CYS | L | 23 | CYS | L | 104 |
| Model | LEU | L | 25 | LEU | L | 85 |
| Model | ASN | L | 27 | THR | L | 85.3 |
| Model | LYS | L | 36 | THR | L | 107 |
| Model | GLN | L | 40 | GLU | L | 105 |
| Model | TRP | L | 41 | GLN | L | 45.3 |
| Model | LYS | L | 42 | ALA | L | 103 |
| Model | GLU | L | 80 | SER | L | 87 |
| Model | THR | L | 83 | SER | L | 85.1 |
| Model | LEU | L | 91 | ASP | L | 95 |
| Model | SER | L | 92 | ASP | L | 95 |
| Model | ALA | L | 103 | SER | L | 120 |
| Model | GLU | L | 105 | THR | L | 118 |
| Model | HIS | L | 108 | LEU | L | 113 |
| Model | ASN | L | 122 | GLU | L | 125 |
| CH1 | | | | | | |
| Model | LYS | H | 1.1 | PHE | H | 29 |
| Model | PRO | H | 2 | TYR | H | 28 |
| Model | PHE | H | 5 | LEU | H | 24 |
| Model | PRO | H | 6 | VAL | H | 121 |
| Model | PRO | H | 9 | ALA | H | 19 |
| Model | PRO | H | 9 | LEU | H | 21 |
| Model | PRO | H | 9 | PRO | H | 123 |
| Native | CYS | H | 23 | CYS | H | 104 |
| Model | LYS | H | 26 | ASP | H | 27 |
| Model | LYS | H | 26 | SER | H | 85.1 |
| Model | ASP | H | 27 | LEU | H | 85.3 |
| Model | GLU | H | 33 | PRO | H | 32 |
| Model | TRP | H | 41 | LEU | H | 45.1 |
| Model | ASN | H | 42 | LEU | H | 45.1 |
| Model | VAL | H | 78 | VAL | H | 89 |
| Model | THR | H | 80 | SER | H | 87 |
| Model | ALA | H | 83 | TYR | H | 85.2 |
| Model | ALA | H | 83 | LEU | H | 85 |
| Model | SER | H | 84.4 | LEU | H | 85.3 |
| Model | PRO | H | 92 | SER | H | 95 |
| Model | VAL | H | 106 | VAL | H | 117 |
| Model | HIS | H | 108 | THR | H | 115 |
| Model | SER | H | 113 | THR | H | 115 |

TABLE 3
Preferred sites of bridge piers of an artificial disulfide
bridge within a constant immunoglobulin domain of
an Fc region, particularly of human origin

| Antibody | | Residue No | | | Residue No |
|---|---|---|---|---|---|
| domain | Residue | IMGT | Residue | Res No | IMGT |
| CH2 | | | | | |
| Model | PRO | 2 | ASP | 265 | 27 |
| Model | PRO | 2 | VAL | 266 | 28 |
| Model | LEU | 6 | LYS | 334 | 119 |
| Model | PHE | 7 | THR | 260 | 22 |
| Model | PRO | 9 | PRO | 257 | 19 |
| Model | LYS | 10 | ASP | 249 | 13 |
| Model | PRO | 11 | ASP | 376 | 34 |
| Model | PRO | 11 | ALA | 378 | 36 |
| Model | LYS | 12 | ALA | 378 | 36 |
| Model | ASP | 13 | ARG | 255 | 17 |
| Model | ASP | 13 | PRO | 257 | 19 |
| Model | THR | 14 | PRO | 257 | 19 |
| Model | LEU | 15 | HIS | 435 | 115 |
| Model | MET | 15.1 | ARG | 255 | 17 |
| Model | MET | 15.1 | SER | 254 | 16 |
| Model | VAL | 21 | LEU | 306 | 89 |
| Model | CYS | 23 | SER | 304 | 87 |
| Native | CYS | 23 | CYS | 321 | 104 |
| Model | VAL | 25 | VAL | 302 | 85 |
| Model | VAL | 26 | ASP | 265 | 27 |
| Model | VAL | 28 | TYR | 300 | 85.2 |
| Model | SER | 29 | ASP | 270 | 32 |
| Model | ASP | 32 | ALA | 327 | 109 |
| Model | LYS | 36 | SER | 324 | 107 |
| Model | ASN | 40 | LYS | 322 | 105 |
| Model | TYR | 42 | LYS | 320 | 103 |
| Model | ALA | 78 | LEU | 306 | 89 |
| Model | THR | 80 | SER | 304 | 87 |
| Model | LYS | 81 | VAL | 303 | 86 |
| Model | ARG | 83 | VAL | 302 | 85 |
| Model | GLU | 84 | ARG | 301 | 85.1 |
| Model | GLN | 84.2 | THR | 299 | 85.3 |
| Model | LEU | 92 | ASP | 312 | 95 |
| Model | ASP | 95 | LYS | 317 | 100 |
| Model | GLU | 101 | SER | 337 | 122 |
| Model | LYS | 103 | THR | 335 | 120 |
| Model | VAL | 106 | ILE | 332 | 117 |
| Model | SER | 107 | PRO | 331 | 116 |
| Model | PRO | 112 | ALA | 330 | 115 |
| Model | ALA | 124 | PRO | 374 | 30 |
| CH3 | | | | | |
| Model | PRO | 1.2 | ALA | 431 | 110 |
| Model | ARG | 1.1 | TYR | 373 | 29 |
| Model | GLU | 1 | ALA | 431 | 110 |
| Model | PRO | 2 | PHE | 372 | 28 |
| Model | VAL | 4 | LYS | 439 | 119 |
| Model | TYR | 5 | LEU | 368 | 24 |
| Model | THR | 6 | LEU | 441 | 121 |
| Model | LEU | 7 | THR | 366 | 22 |
| Model | PRO | 9 | GLU | 357 | 13 |
| Model | PRO | 9 | VAL | 363 | 19 |
| Model | SER | 10 | ASP | 356 | 12 |
| Model | SER | 10 | GLU | 357 | 13 |
| Model | LEU | 21 | LEU | 410 | 89 |
| Model | CYS | 23 | SER | 408 | 87 |
| Native | CYS | 23 | CYS | 425 | 104 |
| Model | LYS | 26 | PHE | 405 | 85.1 |
| Model | SER | 33 | PHE | 404 | 85.2 |
| Model | SER | 33 | PRO | 396 | 83 |
| Model | ALA | 36 | MET | 428 | 107 |
| Model | TRP | 41 | GLU | 388 | 45.3 |
| Model | TYR | 78 | LEU | 410 | 89 |
| Model | THR | 80 | SER | 408 | 87 |
| Model | PRO | 83 | PHE | 404 | 85.2 |
| Model | VAL | 91 | ARG | 416 | 95 |
| Model | ASP | 92 | ARG | 416 | 95 |
| Model | VAL | 101 | SER | 442 | 122 |
| Model | PHE | 102 | LEU | 441 | 121 |
| Model | SER | 103 | SER | 440 | 120 |
| Model | SER | 105 | GLN | 438 | 118 |
| Model | HIS | 108 | LEU | 432 | 112 |

Additional positions which can be mutated in order to create artificial disulfide bonds are for example in the CH1 domain: P6C+K119C, or V4C+V117C, or V25C+V106C; and in the CH3 domain: T6C+K119C, or V4C+K119C (IMGT numbering).

According to a preferred embodiment artificial disulfide bridges were formed with Cys bridge piers introduced at the terminal Fc sequence, such as the C-terminal sequence, which is optionally combined with an artificial disulfide bridge formed by further Cys mutations at positions near the N-terminus of the CH3 domain and the FG loop, and/or combined with an artificial disulfide bridge formed by further Cys mutations at positions in the BC loop and the D sheet.

The preferred sites of mutations are not within the region of a native disulfide bridge to enforce a native disulfide bridge, but apart from the site of a native disulfide bridge. In some cases it may be preferred to engineer at least two artificial disulfide bonds, even at least three artificial disulfide bonds within a modular antibody are feasible.

The modular antibody according to the invention has a surprisingly increased thermostability. Even when a stable format, such as a CH3 antibody domain, or a CH3 dimer or an Fc antibody fragment is used as a source material, it was still possible to significantly increase the thermal stability of the CH3 domain as measured by differential scanning calorimetry (DSC). It was even more surprising that the thermal stability of the CH3 domain within the context of a stabilized Fc fragment according to the invention could be significantly increased, while the denaturation of the CH2 domain remained unchanged. The disulfide stabilization preferably leads to a thermostability increase by at least 5° C., more preferably at least 6° C., or at least 7° C., or at least 8° C., or at least 9° C., or at least 10° C. It turned out that the modular antibody according to the invention with a thermal stability of at least 77° C., preferably at least 78° C., more preferably at least 79° C., more preferably at least 80° C., more preferably at least 81° C., or at least 82° C., or at least 83° C., or at least 84° C., or at least 85° C., or at least 86° C., or at least 87° C., or at least 88° C., or at least 89° C., or at least 90° C., even more than 90° C., possibly up to 100° C., is most preferred. In particular, a preferred Fc fragment stabilized through an artificial disulfide bond was obtained having a melting point (Tm) as determined by DSC of more than 91° C., which corresponds to an increase in stability of more than 9° C. In an antigen binding Fc molecule, which usually would have a lower thermostability than the wild-type, an increase of thermostability could be shown by the method according to the invention. An exemplary modular antibody according to the invention contains an interdomain, e.g. an interchain disulfide bridge, such to connect two heavy chain immunoglobulins. Mutating a few residues within a CH3 domain allows for the formation of a disulfide bridge spanning over the C-terminus of the CH3 pair within an Fc fragment, with or without a hinge region. An exemplary mutant comprises a disulfide bridge, which is structurally and functionally homologous to the disulfide bridge connecting the C-terminus of the CL domain to the CH1 domain in Fab fragments and complete antibodies.

Thus, a stabilized homodimeric immunoglobulin was provided as a scaffold to engineer new binding sites into the loop region of the immunoglobulin. The stabilized scaffold and antigen-binding variants obtained from a respective library may be tested by DSC to assess the thermostability, as determined by the melting point. Variants of a stabilized scaffold turned out to essentially maintain the thermostability of the scaffold. Thus, antigen-binding variants of a thermostable scaffold according to the invention would show an increased thermostability over the respective scaffold without having the additional disulfide bridge.

The modular antibody according to the invention preferably comprises at least one antigen-binding site within the variable and/or the framework region of a variable and/or a constant domain, either formed by CDR loops or within the structural loop region. Thus, the modular according to the present invention optionally exerts one or more binding regions to antigens, including binding sites binding specifically to an epitope of an antigen and binding sites potentially mediating effector function. Binding sites to one or more antigens may be presented by the CDR-region or any other natural receptor binding structure, or be introduced into a structural loop region of an antibody domain, either of a variable or constant domain structure. The antigens as used for testing the binding properties of the binding sites may be naturally occurring molecules or chemically synthesized molecules or recombinant molecules, either in solution or in suspension, e.g. located on or in particles such as solid phases, on or in cells or on viral surfaces. It is preferred that the binding of a modular antibody to an antigen is determined when the antigen is still adhered or bound to molecules and structures in the natural context. Thereby it is possible to identify and obtain those modified modular antibodies that are best suitable for the purpose of diagnostic or therapeutic use.

The stabilized modular antibody according to the invention is particularly useful as a scaffold for mutagenesis to introduce new binding sites. It is possible to use the engineered proteins to produce molecules which are monospecific, bispecific, trispecific, and may even carry more specificities. By the invention it is be possible to provide a stable framework of a modular antibody for a multispecific binding agent.

A multidomain modular antibody according to the invention may be modified within a loop or loop region to provide variants or to provide a new binding site, either within a CDR-loop or a non-CDR loop, structural loops of a constant domain being the preferred sites of modifications or mutagenesis.

It is preferred to modify at least one loop region of a modular antibody according to the invention, which results in a substitution, deletion and/or insertion of one or more nucleotides or amino acids, preferably a point mutation, or even the exchange of whole loops, more preferred the change of at least 2, 3, 4, 5, 6, 7, 8, 9, 10, 11, 12, 13, 14 or 15, up to 30 amino acids. Thereby the modified sequence comprises amino acids not included in the conserved regions of the loops, the newly introduced amino acids being naturally occurring, but foreign to the site of modification, or substitutes of naturally occurring amino acids.

However, the maximum number of amino acids inserted into a loop region of a binding agent preferably may not exceed the number of 30, preferably 25, more preferably 20 amino acids at a maximum. The substitution and the insertion of the amino acids occurs preferably randomly or semi-randomly using all possible amino acids or a selection of preferred amino acids for randomization purposes, by methods known in the art and as disclosed in the present patent application.

The site of modification may be at a specific single loop or a loop region, in particular a structural loop or a structural loop region. A loop region usually is composed of at least two, preferably at least 3 or at least 4 loops that are adjacent to each other, and which may contribute to the binding of an antigen through forming an antigen binding site or antigen binding pocket. It is preferred that the one or more sites of modification are located within the area of 10 amino acids, more preferably within 20, 30, 40, 50, 60, 70, 80, 90 up to 100 amino acids, in particular within a structural region to form a surface or pocket where the antigen can sterically access the loop regions.

In this regard the preferred modifications are engineered in the loop regions of CH1, CH2, CH3 and CH4, in particular in the range of amino acids 7 to 21, amino acids 25 to 39, amino acids 41 to 81, amino acids 83 to 85, amino acids 89 to 103 and amino acids 106 to 117.

In another preferred embodiment a modification in the structural loop region comprising amino acids 92 to 98 is combined with a modification in the structural loop region comprising amino acids 8 to 20.

The above identified amino acid regions of the respective immunoglobulins comprise loop regions to be modified. Preferably, a modification in the structural loop region comprising amino acids 92 to 98 is combined with a modification in one or more of the other structural loops.

In a preferred embodiment a modification in the structural loop region comprising amino acids 92 to 98 is combined with a modification in the structural loop region comprising amino acids 41 to 45.2.

Most preferably each of the structural loops comprising amino acids 92 to 98, amino acids 41 to 45.2 and amino acids 8 to 20 contain at least one amino acid modification.

In another preferred embodiment each of the structural loops comprising amino acids 92 to 98, amino acids 41 to 45.2, and amino acids 8 to 20 contain at least one amino acid modification.

According to another preferred embodiment the amino acid residues in the area of positions 15 to 17, 29 to 34, 41 to 45.2, 84 to 85, 92 to 100, and/or 108 to 115 of CH3 are modified.

The preferred modifications of Igk-C and Igl-C of human origin are engineered in the loop regions in the area of amino acids 8 to 20, amino acids 26 to 36, amino acids 41 to 82, amino acids 83 to 88, amino acids 92 to 100, amino acids 107 to 124 and amino acids 123 to 126.

The preferred modifications of loop regions of Igk-C and Igl-C of murine origin are engineered at sites in the area of amino acids 8 to 20, amino acids 26 to 36, amino acids 43 to 79, amino acids 83 to 85, amino acids 90 to 101, amino acids 108 to 116 and amino acids 122 to 126.

Another preferred modular antibody of the invention consists of a variable domain of a heavy or light chain, or a part thereof including a minidomain, having at least one framework and one loop region, preferably a structural loop region, which is characterised in that said at least one loop region comprises at least one amino acid modification forming at least one modified loop region, wherein said at least one modified loop region forms a relevant binding site as described above.

Accordingly, an immunoglobulin preferably used according to the invention may contain a modification within the variable domain, which is selected from the group of VH, Vkappa, Vlambda, VHH and combinations thereof. More specifically, they comprise at least one modification within amino acids 7 to 22, amino acids 39 to 55, amino acids 66 to 79, amino acids 77 to 89 or amino acids 89 to 104, where the numbering of the amino acid position of the domains is that of the IMGT.

In a specific embodiment, the immunoglobulin preferably used according to the invention is characterised in that the loop regions of VH or Vkappa or Vlambda of human origin comprise at least one modification within amino acids 7 to 22, amino acids 43 to 51, amino acids 67 to 77, amino acids 77 to 88, and amino acids 89 to 104, most preferably amino acid positions 12 to 17, amino acid positions 45 to 50, amino acid positions 68 to 77, amino acids 79 to 88, and amino acid positions 92 to 99, where the numbering of the amino acid position of the domains is that of the IMGT.

The structural loop regions of the variable domain of the immunoglobulin of human origin, as possible selected for modification purposes are preferably located in the area of amino acids 8 to 20, amino acids 44 to 50, amino acids 67 to 76, amino acids 78 to 87, and amino acids 89 to 101.

According to a preferred embodiment the structural loop regions of the variable domain of the immunoglobulin of murine origin as possible selected for modification purposes are preferably located in the area of amino acids 6 to 20, amino acids 43 to 52, amino acids 67 to 79, amino acids 79 to 87, and amino acids 91 to 100.

A preferred method according to the invention refers to a randomly modified nucleic acid molecule coding for an immunoglobulin, immunoglobulin domain or a part thereof which comprises at least one nucleotide repeating unit within a structural loop coding region having the sequence 5′-NNS-3′,5′-NNN-3′,5′-NNB-3′ or 5′-NNK-3′. In some embodiments the modified nucleic acid comprises nucleotide codons selected from the group of TMT, WMT, BMT, RMC, RMG, MRT, SRC, KMT, RST, YMT, MKC, RSA, RRC, NNK, NNN, NNS or any combination thereof (the coding is according to IUPAC).

The modification of the nucleic acid molecule may be performed by introducing synthetic oligonucleotides into a larger segment of nucleic acid or by de novo synthesis of a complete nucleic acid molecule. Synthesis of nucleic acid may be performed with tri-nucleotide building blocks which would reduce the number of nonsense sequence combinations if a subset of amino acids is to be encoded (e.g. Yanez et al. Nucleic Acids Res. (2004) 32:e158; Virnekas et al. Nucleic Acids Res. (1994) 22:5600-5607).

Another important aspect of the invention is that each potential binding domain remains physically associated with the particular DNA or RNA molecule which encodes it, and in addition, a the fusion proteins oligomerize at the surface of a genetic package to present the binding polypeptide in the native and functional oligomeric structure. Once successful binding domains are identified, one may readily obtain the gene for expression, recombination or further engineering purposes. The form that this association takes is a “replicable genetic package”, such as a virus, cell or spore which replicates and expresses the binding domain-encoding gene, and transports the binding domain to its outer surface. Another form is an in-vitro replicable genetic package such as ribosomes that link coding RNA with the translated protein. In ribosome display the genetic material is replicated by enzymatic amplification with polymerases.

Those cells or viruses or nucleic acid bearing the binding agents which recognize the target molecule are isolated and, if necessary, amplified. The genetic package preferably is M13 phage, and the protein includes the outer surface transport signal of the M13 gene III protein.

The preferred expression system for the fusion proteins is a non-suppressor host cell, which would be sensitive to a stop codon, such as an amber stop codon, and would thus stop translation thereafter. In the absence of such a stop codon such non-suppressor host cells, preferably E. coli, are preferably used. In the presence of such a stop codon supressor host cells would be used.

Preferably in the method of this invention the vector or plasmid of the genetic package is under tight control of the transcription regulatory element, and the culturing conditions are adjusted so that the amount or number of vector or phagemid particles displaying less than two copies of the fusion protein on the surface of the particle is less than about 20%. More preferably, the amount of vector or phagemid particles displaying less than two copies of the fusion protein is less than 10% the amount of particles displaying one or more copies of the fusion protein. Most preferably the amount is less than 1%.

The expression vector preferably used according to the invention is capable of expressing a binding polypeptide, and may be produced as follows: First a binding polypeptide gene library is synthesized by introducing a plurality of polynucleotides encoding different binding sequences. The plurality of polynucleotides may be synthesized in an appropriate amount to be joined in operable combination into a vector that can be propagated to express a fusion protein of said binding polypeptide. Alternatively the plurality of olynucleotides can also be amplified by polymerase chain reaction to obtain enough material for expression. However, this would only be advantageous if the binding polypeptide would be encoded by a large polynucleotide sequence, e.g. longer than 200 base pairs or sometimes longer than 300 base pairs. Thus, a diverse synthetic library is preferably formed, ready for selecting from said diverse library at least one expression vector capable of producing binding polypeptides having the desired preselected function and binding property, such as specificity.

The randomly modified nucleic acid molecule may comprise the above identified repeating units, which code for all known naturally occurring amino acids or a subset thereof. Those libraries that contain modified sequences wherein a specific subset of amino acids are used for modification purposes are called “focused” libraries. The member of such libraries have an increased probability of an amino acid of such a subset at the modified position, which is at least two times higher than usual, preferably at least 3 times or even at least 4 times higher. Such libraries have also a limited or lower number of library members, so that the number of actual library members reaches the number of theoretical library members. In some cases the number of library members of a focused library is not less than 10³ times the theoretical number, preferably not less than 10² times, most preferably not less than 10 times.

The modular antibody according to the invention is particularly useful as a stable scaffold for a library preparation. It is understood that the term “library of modular antibodies” always includes libraries of proteins, fusion proteins, genetic packages or nucleic acids encoding such variants of a modular antibody, which are members of a library.

The term “fusion protein” or “chimeric fusion protein” as used for the purpose of the invention shall mean the molecule composed of a genetic package, at least part of an outer surface structure, such as a coat protein, optionally a linker sequence, and a binding agent. The fusion protein is encoded by a vector with the gene of the binding agent and information to display a copy of the binding agent at the surface of the genetic package.

Variants of said scaffold are preferably produced by mutagenesis in those parts of the molecule that are not involved in the artificial disulfide bond, e.g. preferably within the loop region or within the C-terminal or N-terminal region.

Methods for production and screening of antibody variants are well-known in the art. General methods for antibody molecular biology, expression, purification, and screening are also well-known in the art.

A library according to the invention may be designed as a dedicated library that contains at least 50% specific formats, preferably at least 60%, more preferred at least 70%, more preferred at least 80%, more preferred at least 90%, or those that mainly consist of specific antibody formats. Specific antibody formats are preferred, such that the preferred library according to the invention it is selected from the group consisting of a VH library, VHH library, Vkappa library, Vlambda library, Fab library, a CH1/CL library, an Fc library and a CH3 library. Libraries characterized by the content of composite molecules containing more than one antibody domains, such as an IgG library or Fc library are specially preferred. Other preferred libraries are those containing T-cell receptors, forming T-cell receptor libraries. Further preferred libraries are epitope libraries, wherein the fusion protein comprises a molecule with a variant of an epitope, also enabling the selection of competitive molecules having similar binding function, but different functionality. Exemplary is a TNFalpha library, wherein trimers of the TNFalpha fusion protein are displayed by a single genetic package.

Another important aspect of the invention is that each potential binding domain remains physically associated with the particular DNA or RNA molecule which encodes it, and in addition, a the fusion proteins oligomerize at the surface of a genetic package to present the binding polypeptide in the native and functional oligomeric structure. Once successful binding domains are identified, one may readily obtain the gene for expression, recombination or further engineering purposes. The form that this association takes is a replicable genetic packag”, such as a virus, cell or spore which replicates and expresses the binding domain-encoding gene, and transports the binding domain to its outer surface. Another form is an in-vitro replicable genetic package such as ribosomes that link coding RNA with the translated protein. In ribosome display the genetic material is replicated by enzymatic amplification with polymerases.

Those cells or viruses or nucleic acid bearing the binding agents which recognize the target molecule are isolated and, if necessary, amplified. The preferred expression system for the fusion proteins is a non-suppressor host cell, which would be sensitive to a stop codon, such as an amber stop codon, and would thus stop translation thereafter. In the absence of such a stop codon such non-suppressor host cells, preferably E. coli, are preferably used. In the presence of such a stop codon supressor host cells would be used.

Preferably in the method of this invention the vector or plasmid of the genetic package is under tight control of the transcription regulatory element, and the culturing conditions are adjusted so that the amount or number of vector or phagemid particles displaying less than two copies of the fusion protein on the surface of the particle is less than about 20%. More preferably, the amount of vector or phagemid particles displaying less than two copies of the fusion protein is less than 10% the amount of particles displaying one or more copies of the fusion protein. Most preferably the amount is less than 1%.

The expression vector preferably used according to the invention is capable of expressing a binding polypeptide, and may be produced as follows: First a binding polypeptide gene library is synthesized by introducing a plurality of polynucleotides encoding different binding sequences. The plurality of polynucleotides may be synthesized in an appropriate amount to be joined in operable combination into a vector that can be propagated to express a fusion protein of said binding polypeptide. Alternatively the plurality of olynucleotides can also be amplified by polymerase chain reaction to obtain enough material for expression. However, this would only be advantageous if the binding polypeptide would be encoded by a large polynucleotide sequence, e.g. longer than 200 base pairs or sometimes longer than 300 base pairs. Thus, a diverse synthetic library is preferably formed, ready for selecting from said diverse library at least one expression vector capable of producing binding polypeptides having the desired preselected function and binding property, such as specificity.

Various alternatives are available for the manufacture of genes encoding the randomized library. It is possible to produce the DNA by a completely synthetic approach, in which the sequence is divided into overlapping fragments which are subsequently prepared as synthetic oligonucleotides. These oligonucleotides are mixed together, and annealed to each other by first heating to ca. 100° C. and then slowly cooling down to ambient temperature. After this annealing step, the synthetically assembled gene can be either cloned directly, or it can be amplified by PCR prior to cloning.

Alternatively, other methods for site directed mutagenesis can be employed for generation of the library insert, such as the Kunkel method (Kunkel TA. Rapid and efficient site-specific mutagenesis without phenotypic selection. Proc Natl Acad Sci U S A. 1985 January; 82(2):488-92) or the Dpnl method (Weiner M P, Costa G L, Schoettlin W, Cline J, Mathur E, Bauer J C. Site-directed mutagenesis of double-stranded DNA by the polymerase chain reaction. Gene. 1994 Dec. 30; 151(1-2):119-23.).

For various purposes, it may be advantageous to introduce silent mutations into the sequence encoding the library insert. For example, restriction sites can be introduced which facilitate cloning or modular exchange of parts of the sequence. Another example for the introduction of silent mutations is the ability to “mark” libraries, that means to give them a specific codon at a selected position, allowing them (or selected clones derived from them) e.g. to be recognized during subsequent steps, in which for example different libraries with different characteristics can be mixed together and used as a mixture in the panning procedure.

An appropriate scaffold ligand may be used for the quality control of a library of modular antibodies according to the invention. The scaffold ligand can be selected from the group consisting of an effector molecule, FcRn, Protein A, Protein G, Protein L and CDR target. As an example, the effector molecule can be selected from the group consisting of CD64, CD32, CD16, Fc receptors.

The method according to the invention can provide a library containing at least 10² independent clones expressing functional oligomers of modular antibody domains or variants thereof. According to the invention it is also provided a pool of preselected independent clones, which is e.g. affinity maturated, which pool comprises preferably at least 10, more preferably at least 100, more preferably at least 1000, more preferably at least 10000, even more than 100000 independent clones. Those libraries, which contain the preselected pools, are preferred sources to select the high affinity modular antibodies according to the invention.

Libraries as used according to the invention preferably comprise at least 10² library members, more preferred at least 10³, more preferred at least 10⁴, more preferred at least 10⁵, more preferred at least 10⁶ library members, more preferred at least 10⁷, more preferred at least 10⁸, more preferred at least 10⁹, more preferred at least 10¹⁰, more preferred at least 10¹¹, up to 10¹² members of a library, preferably derived from a parent molecule, which is a functional modular antibody as a scaffold containing at least one specific function or binding moiety, and derivatives thereof to engineer a new binding site apart from the original, functional binding region of said parent moiety.

Usually the libraries according to the invention further contain variants of the modular antibody according to the invention, resulting from mutagenesis or randomization techniques. These variants include inactive or non-functional antibodies. Thus, it is preferred that any such libraries be screened with the appropriate assay for determining the functional effect. Preferred libraries, according to the invention, comprise at least 10² variants of modular antibodies, more preferred at least 10³, more preferred at least 10⁴, more preferred at least 10⁵, more preferred at least 10⁶, more preferred at least 10⁷, more preferred at least 10⁸, more preferred at least 10⁹, more preferred at least 10¹⁰, more preferred at least 10¹¹, up to 10¹² variants or higher to provide a highly diverse repertoire of antibodies for selecting the best suitable binders. Any such synthetic libraries may be generated using mutagenesis methods as disclosed herein.

As is well-known in the art, there is a variety of display and selection technologies that may be used for the identification and isolation of proteins with certain binding characteristics and affinities, including, for example, display technologies such as cellular and non-cellular methods, in particular mobilized display systems. Among the cellular systems the phage display, virus display, yeast or other eukaryotic cell display, such as mammalian or insect cell display, may be used. Mobilized systems are relating to display systems in the soluble form, such as in vitro display systems, among them ribosome display, mRNA display or nucleic acid display.

Preferably the library is a yeast library and the yeast host cell exhibits at the surface of the cell the oligomers with the biological activity. The yeast host cell is preferably selected from the genera Saccharomyces, Pichia, Hansenula, Schizisaccharomyces, Kluyveromyces, Yarrowia and Candida. Most preferred, the host cell is Saccharomyces cerevisiae.

The preferred method of producing the modular antibody according to the invention refers to engineering a modular antibody that is binding specifically to at least one first epitope and comprising modifications in each of at least two structural loop regions, and determining the specific binding of said at least two loop regions to at least one second epitope, wherein the unmodified structural loop region (non-CDR region) does not specifically bind to said at least one second epitope. Thus, an antibody or antigen-binding structure specific for a first antigen may be improved by adding another valency or specificity against a second antigen, which specificity may be identical, either targeting different epitopes or the same epitope, to increase valency or to obtain bi-, oligo- or multispecific molecules.

The modular antibody according to the invention preferably comprises a binding site to act as a binding agent or binding partner.

For the purposes of this invention, the term “binding agent” or “ligand” refers to a member of a binding pair, in particular binding polypeptides having the potential of serving as a binding domain for a binding partner. Examples of binding partners include pairs of binding agents with functional interactions, such as receptor binding to ligands, antibody binding to antigen or receptors, a drug binding to a target, and enzyme binding to a substrate.

Binding partners are agents that specifically bind to one another, usually through non-covalent interactions. Examples of binding partners include pairs of binding agents with functional interactions, such as receptor binding to ligands, antibody binding to antigen, a drug binding to a target, and enzyme binding to a substrate. Binding partners have found use in many therapeutic, diagnostic, analytical and industrial applications. Most prominent binding pairs are antibodies or immunoglobulins, fragments or derivatives thereof. In most cases the binding of such binding agents is required to mediate a biological effect or a function, a “functional interaction”.

According to a specific embodiment of the present invention the modular antibody according to the invention is an immunoglobulin of human or murine origin, and may be employed for various purposes, in particular in pharmaceutical compositions. Of course, the modular antibody according to the invention may also be a humanized or chimeric immunoglobulin.

The modular antibody according to the invention, which is a human immunoglobulin, is preferably selected or derived from the group consisting of IgA1, IgA2, IgD, IgE, IgG1, IgG2, IgG3, IgG4 and IgM. The murine immunoglobulin according to the invention is preferably selected or derived from the group consisting of IgA, IgD, IgE, IgG1, IgG2A, IgG2B, IgG2C, IgG3 and IgM.

Preferably the modular antibody according to the invention is glycosylated. More preferably the glycosylation is a eukaryotic or plant glycosylation, such as a human, yeast or moss glycosylation.

The modular antibodies according to the invention can be used as isolated polypeptides or as combination molecules, e.g. through recombination, fusion or conjugation techniques, with other peptides or polypeptides. The peptides are preferably homologous to immunoglobulin domain sequences, and are preferably at least 5 amino acids long, more preferably at least 10 or even at least 50 or 100 amino acids long, and constitute at least partially the loop region of the immunoglobulin domain. The preferred binding characteristics relate to predefined epitope binding, affinity and avidity.

The engineered molecules according to the present invention will be useful as stand-alone molecules, as well as fusion proteins or derivatives, most typically fused before or after modification in such a way as to be part of larger structures, e.g. of complete antibody molecules, or parts thereof. Immunoglobulins or fusion proteins as produced according to the invention thus also comprise Fc fragments, Fab fragments, Fv fragments, single chain antibodies, in particular single-chain Fv fragments, bi- or multispecific scFv, diabodies, unibodies, multibodies, multivalent or multimers of immunoglobulin domains and others.

The modular antibody according to the invention is possibly further combined with one or more modified modular antibodies or with unmodified modular antibodies, or parts thereof, to obtain a combination modular antibody. Combinations are preferably obtained by recombination techniques, but also by binding through adsorption, electrostatic interactions or the like, or else through conjugation or chemical binding with or without a linker. The preferred linker sequence is either a natural linker sequence or functionally suitable artificial sequence.

In general the modular antibody according to the invention may be used as a building block to molecularly combine other modular antibodies or biologically active substances or molecules. It is preferred to molecularly combine at least one antibody binding to the specific partner via the variable or non-variable sequences, like structural loops, with at least one other binding molecule which can be an antibody, antibody fragment, a soluble receptor, a ligand or another antibody domain, or a binding moiety thereof. Other combinations refer to proteinaceous molecules, nucleic acids, lipids, organic molecules and carbohydrates.

It is preferred to make use of those modular antibodies according to the invention that contain native structures interacting with effector molecules or immune cells, thus providing for ADCC, CDC or ADPC. Those native structures either remain unchanged or are modulated for an increased effector function. Binding sites for e.g. Fc receptors are described to be located in a CH2 and/or CH3 domain region, and may be mutagenized by well known techniques.

ADCC, antibody-dependent cell-mediated cytotoxicity is the killing of antibody-coated target cells by cells with Fc receptors that recognize the constant region of the bound antibody. Most ADCC is mediated by NK cells that have the Fc receptor FcgammaRIII or CD16 on their surface. Typical assays employ target cells, like Ramos cells, incubated with serially diluted antibody prior to the addition of freshly isolated effector cells. The ADCC assay is then further incubated for several hours and % cytotoxicity detected. Usually the Target: Effector ratio is about 1:16, but may be 1:1 up to 1:50.

Complement-dependent cytotoxicity (CDC) is a mechanism of killing cells in which antibody bound to the target cell surface fixes complement, which results in assembly of the membrane attack complex that punches holes in the target cell membrane resulting in subsequent cell lysis. The commonly used CDC assay follows the same procedure as for ADCC determination, however, with complement containing serum instead of effector cells.

The modular antibody according to the invention preferably has a cytotoxic activity as determined by either of ADCC and CDC assay, preferably in a way to provide a significant increase in the percentage of cytolysis as compared to a control. The absolute percentage increase preferably is higher than 5%, more preferably higher than 10%, even more preferred higher than 20%.

The antibody-dependent cellular phagocytosis, ADCP sometimes called ADPC, is usually investigated side by side with cytolysis of cultured human cells. Phagocytosis by phagocytes, usually human monocytes or monocyte-derived macrophages, as mediated by an antibody can be determined as follows. Purified monocytes may be cultured with cytokines to enhance expression of FcγRs or to induce differentiation into macrophages. ADCP and ADCC assays are then performed with target cells. Phagocytosis is determined as the percentage of positive cells measured by flow cytometry. The positive ADCP activity is proven with a significant uptake of the antibody-antigen complex by the phagocytes. The absolute percentage preferably is higher than 5%, more preferably higher than 10%, even more preferred higher than 20%.

In a typical assay PBMC or monoycytes or monocyte derived macrophages are resuspended in RF2 medium (RPMI 1640 supplemented with 2% FCS) in 96-well plates at a concentration of 1×10⁵ viable cells in 100 ml/well. Appropriate target cells, expressing the target antigen, e.g. Her2/neu antigen and SKBR3 cells, are stained with PKH2 green fluorescence dye. Subsequently 1×10⁴ PKH2-labeled target cells and an Her 2 specific (IgG1) antibody (or modular antibody) or mouse IgG1 isotype control (or modular antibody control) are added to the well of PBMC's in different concentrations (e.g. 1-100 μg/ml) and incubated in a final volume of 200 ml at 37° C. for 24 h. Following the incubation, PBMCs or monoycytes or monocyte derived macrophages and target cells are harvested with EDTA-PBS and transferred to 96-well V-bottomed plates. The plates are centrifuged and the supernatant is aspirated. Cells are counterstained with a 100-ml mixture of RPE-conjugated anti-CD11b, anti-CD14, and human IgG, mixed and incubated for 60 min on ice. The cells are washed and fixed with 2% formaldehyde-PBS. Two-color flow cytometric analysis is performed with e.g. a FACS Calibur under optimal gating. PKH2-labeled target cells (green) are detected in the FL-1 channel (emission wavelength, 530 nm) and RPE-labeled PBMC or monoycytes or monocyte derived macrophages (red) are detected in the FL-2 channel (emission wavelength, 575 nm). Residual target cells are defined as cells that are PKH2⁺/RPE⁻ Dual-labeled cells (PKH2⁺/RPE⁻) are considered to represent phagocytosis of targets by PBMC or monoycytes or monocyte derived macrophages. Phagocytosis of target cells is calculated with the following equation: percent phagocytosis=100×[(percent dual positive)/(percent dual positive+percent residual targets)]. All tests are usually performed in duplicate or triplicate and the results are expressed as mean 6 SD.

The preferred effector function of the modular antibody according to the invention usually differs from any synthetic cytotoxic activity, e.g. through a toxin that may be conjugated to an immunoglobulin structure. Toxins usually do not activate effector molecules and the biological defence mechanism. Thus, the preferred cytotoxic activity of the modular antibodies according to the invention is a biological cytotoxic activity, which usually is immunostimulatory, leading to effective cytolysis.

The modular antibody according to the invention may specifically bind to any kind of binding molecules or structures, in particular to antigens, proteinaceous molecules, proteins, peptides, polypeptides, nucleic acids, glycans, carbohydrates, lipids, organic molecules, in particular small organic molecules, anorganic molecules, or combinations or fusions thereof, including PEG, prodrugs or drugs. The preferred modular antibody according to the invention may comprise at least two loops or loop regions whereby each of the loops or loop regions may specifically bind to different molecules or epitopes.

According to a further preferred embodiment the target antigen is selected from those antigens presented by cells, e.g. cellular targets, like epithelial cells, cells of solid tumors, infected cells, blood cells, antigen-presenting cells and mononuclear cells.

Preferably the target antigen is selected from cell surface antigens, including receptors, in particular from the group consisting of erbB receptor tyrosine kinases (such as EGFR, HER2, HER3 and HER4, in particular those epitopes of the extracellular domains of such receptors, e.g. the 4D5 epitope), molecules of the TNF-receptor superfamily, such as Apo-1 receptor, TNFR1, TNFR2, nerve growth factor receptor NGFR, CD40, T-cell surface molecules, T-cell receptors, T-cell antigen OX40, TACI-receptor, BCMA, Apo-3, DR4, DR5, DR6, decoy receptors, such as DcR1, DcR2, CAR1, HVEM, GITR, ZTNFR-5, NTR-1, TNFL1 but not limited to these molecules, B-cell surface antigens, such as CD10, CD19, CD20, CD21, CD22, antigens or markers of solid tumors or hematologic cancer cells, cells of lymphoma or leukaemia, other blood cells including blood platelets, but not limited to these molecules.

Those antigens are preferably targeted, which are selected from the group consisting of tumor associated antigens, in particular EpCAM, tumor-associated glycoprotein-72 (TAG-72), tumor-associated antigen CA 125, Prostate specific membrane antigen (PSMA), High molecular weight melanoma-associated antigen (HMW-MAA), tumor-associated antigen expressing Lewis Y related carbohydrate, Carcinoembryonic antigen (CEA), CEACAM5, HMFG PEM, mucin MUC1, MUC18 and cytokeratin tumor-associated antigen, bacterial antigens, viral antigens, allergens, allergy related molecules IgE, cKIT and Fc-epsilon-receptorI, IRp60, IL-5 receptor, CCR3, red blood cell receptor (CR1), human serum albumin, mouse serum albumin, rat serum albumin, Fc receptors, like neonatal Fc-gamma-receptor FcRn, Fc-gamma-receptors Fc-gamma RI, Fc-gamma-RII, Fc-gamma RIII, Fc-alpha-receptors, Fc-epsilon-receptors, fluorescein, lysozyme, toll-like receptor 9, erythropoietin, CD2, CD3, CD3E, CD4, CD11, CD11a, CD14, CD16, CD18, CD19, CD20, CD22, CD23, CD25, CD28, CD29, CD30, CD32, CD33 (p67 protein), CD38, CD40, CD40L, CD52, CD54, CD56, CD64, CD80, CD147, GD3, IL-1, IL-1R, IL-2, IL-2R, IL-4, IL-5, IL-6, IL-6R, IL-8, IL-12, IL-15, IL-17, IL-18, IL-23, LIF, OSM, interferon alpha, interferon beta, interferon gamma; TNF-alpha, TNFbeta2, TNFalpha, TNFalphabeta, TNF-R1, TNF-RII, FasL, CD27L, CD30L, 4-1BBL, TRAIL, RANKL, TWEAK, APRIL, BAFF, LIGHT, VEG1, OX40L, TRAIL Receptor-1, A1 Adenosine Receptor, Lymphotoxin Beta Receptor, TACI, BAFF-R, EPO; LFA-3, ICAM-1, ICAM-3, integrin beta1, integrin beta2, integrin alpha4/beta7, integrin alpha2, integrin alpha3, integrin alpha4, integrin alpha5, integrin alpha6, integrin alphav, alphaVbeta3 integrin, FGFR-3, Keratinocyte Growth Factor, GM-CSF, M-CSF, RANKL, VLA-1, VLA-4, L-selectin, anti-Id, E-selectin, HLA, HLA-DR, CTLA-4, T cell receptor, B7-1, B7-2, VNRintegrin, TGFbeta1, TGFbeta2, eotaxin1, BLyS (B-lymphocyte Stimulator), complement C5, IgE, IgA, IgD, IgM, IgG, factor VII, CBL, NCA 90, EGFR (ErbB-1), Her2/neu (ErbB-2), Her3 (ErbB-3), Her4 (ErbB4), Tissue Factor, VEGF, VEGFR, endothelin receptor, VLA-4, carbohydrates such as blood group antigens and related carbohydrates, Galili-Glycosylation, Gastrin, Gastrin receptors, tumor associated carbohydrates, Hapten NP-cap or NIP-cap, T cell receptor alpha/beta, E-selectin, P-glycoprotein, MRP3, MRPS, glutathione-S-transferase pi (multi drug resistance proteins), alpha-granule membrane protein(GMP) 140, digoxin, placental alkaline phosphatase (PLAP) and testicular PLAP-like alkaline phosphatase, transferrin receptor, Heparanase I, human cardiac myosin, Glycoprotein IIb/IIIa (GPIIb/IIIa), human cytomegalovirus (HCMV) gH envelope glycoprotein, HIV gp120, HCMV, respiratory syncital virus RSV F, RSVF Fgp, VNRintegrin, Hep B gp120, CMV, gpIIbIIIa, HIV IIIB gp120 V3 loop, respiratory syncytial virus (RSV) Fgp, Herpes simplex virus (HSV) gD glycoprotein, HSV gB glycoprotein, HCMV gB envelope glycoprotein, Clostridium perfringens toxin and fragments thereof.

Preferred modular antibodies according to the invention are binding said target antigen with a high affinity, in particular with a high on and/or a low off rate, or a high avidity of binding. Usually a binder is considered a high affinity binder with a Kd of less than 10⁻⁹ M. Medium affinity binders with a Kd of less than 10⁻⁶ up to 10⁻⁹ M may be provided according to the invention as well, preferably in conjunction with an affinity maturation process.

Affinity maturation is the process by which antibodies with increased affinity for antigen are produced. With structural changes of an antibody, including amino acid mutagenesis or as a consequence of somatic mutation in immunoglobulin gene segments, variants of a binding site to an antigen are produced and selected for greater affinities. Affinity matured modular antibodies may exhibit a several logfold greater affinity than a parent antibody. Single parent antibodies may be subject to affinity maturation. Alternatively pools of modular antibodies with similar binding affinity to the target antigen may be considered as parent structures that are varied to obtain affinity matured single antibodies or affinity matured pools of such antibodies.

The preferred affinity maturated variant of a modular antibody according to the invention exhibits at least a 10 fold increase in affinity of binding, preferably at least a 100 fold increase. The affinity maturation may be employed in the course of the selection campaigns employing respective libraries of parent molecules, either with modular antibodies having medium binding affinity to obtain a preferred modular antibody of the invention having a high specific target binding property of a Kd<10⁻⁸ M and/or a potency of EC50<10⁻⁸ M. The binding potency or affinity may be even more increased by affinity maturation of the modular antibody according to the invention to obtain the high values corresponding to a Kd or EC50 of less than 10⁻⁹ M, preferably less than 10⁻¹⁰ M or even less than 10⁻¹¹ M, most preferred in the picomolar range.

The EC50, sometimes called IC50, also called 50% saturation concentration, is a measure for the binding potency of a modular antibody. It is the molar concentration of a binder, which produces 50% of the maximum possible binding at equilibrium or under saturation. The potency of an antagonist is usually defined by its IC50 value. This can be calculated for a given antagonist by determining the concentration of antagonist needed to elicit half saturation of the maximum binding of an agonist. Elucidating an IC50 value is useful for comparing the potency of antibodies or antibody variants with similar efficacies; however the dose-response curves produced by both drug antagonists must be similar. The lower the IC50, the greater the potency of the antagonist, and the lower the concentration of drug that is required to inhibit the maximum biological response, like effector function or cytotoxic activity. Lower concentrations of drugs may also be associated with fewer side effects.

Usually the affinity of an antibody correlates well with the IC50. The affinity of an antagonist for its binding site (K), is understood as its ability to bind to a receptor, which determines the duration of binding and respective agonist activity. Measures to increase the affinity by affinity maturation usually also increase the potency of binding, resulting in the respective reduction of IC50 values in the same range of the Kd values.

The IC50 and Kd values may be determined using the saturation binding assays well-known in the art.

The modular antibody according to the invention is preferably conjugated to a label or reporter molecule, selected from the group consisting of organic molecules, enzyme labels, radioactive labels, colored labels, fluorescent labels, chromogenic labels, luminescent labels, haptens, digoxigenin, biotin, metal complexes, metals, colloidal gold and mixtures thereof. Modified immunoglobulins conjugated to labels or reporter molecules may be used, for instance, in assay systems or diagnostic methods.

The modular antibody according to the invention may be conjugated to other molecules which allow the simple detection of said conjugate in, for instance, binding assays (e.g. ELISA) and binding studies.

In a preferred embodiment, antibody variants are screened using one or more cell-based or in vivo assays. For such assays, purified or unpurified modified immunoglobulins are typically added exogenously such that cells are exposed to individual immunoglobulins or pools of immunoglobulins belonging to a library. These assays are typically, but not always, based on the function of the immunoglobulin; that is, the ability of the antibody to bind to its target and mediate some biochemical event, for example effector function, ligand/receptor binding inhibition, apoptosis, and the like. Such assays often involve monitoring the response of cells to the antibody, for example cell survival, cell death, change in cellular morphology, or transcriptional activation such as cellular expression of a natural gene or reporter gene. For example, such assays may measure the ability of antibody variants to elicit ADCC, ADCP, or CDC. For some assays additional cells or components, that is in addition to the target cells, may need to be added, for example example serum complement, or effector cells such as peripheral blood monocytes (PBMCs), NK cells, macrophages, and the like. Such additional cells may be from any organism, preferably humans, mice, rat, rabbit, and monkey. Modular antibodies may cause apoptosis of certain cell lines expressing the target, or they may mediate attack on target cells by immune cells which have been added to the assay. Methods for monitoring cell death or viability are known in the art, and include the use of dyes, immunochemical, cytochemical, and radioactive reagents. For example, caspase staining assays may enable apoptosis to be measured, and uptake or release of radioactive substrates or fluorescent dyes such as alamar blue may enable cell growth or activation to be monitored.

In a preferred embodiment, the DELFIART EuTDA-based cytotoxicity assay (Perkin Elmer, Mass.) may be used. Alternatively, dead or damaged target cells may be monitored by measuring the release of one or more natural intracellular components, for example lactate dehydrogenase.

Transcriptional activation may also serve as a method for assaying function in cell-based assays. In this case, response may be monitored by assaying for natural genes or immunoglobulins which may be upregulated, for example the release of certain interleukins may be measured, or alternatively readout may be via a reporter construct. Cell-based assays may also involve the measure of morphological changes of cells as a response to the presence of modular antibodies. Cell types for such assays may be prokaryotic or eukaryotic, and a variety of cell lines that are known in the art may be employed. Alternatively, cell-based screens are performed using cells that have been transformed or transfected with nucleic acids encoding the variants. That is, antibody variants are not added exogenously to the cells. For example, in one embodiment, the cell-based screen utilizes cell surface display. A fusion partner can be employed that enables display of modified immunoglobulins on the surface of cells (Witrrup, 2001, Curr Opin Biotechnol, 12:395-399).

In a preferred embodiment, the immunogenicity of the modular antibodies may be determined experimentally using one or more cell-based assays. In a preferred embodiment, ex vivo T-cell activation assays are used to experimentally quantitate immunogenicity. In this method, antigen presenting cells and naive T cells from matched donors are challenged with a peptide or whole antibody of interest one or more times. Then, T cell activation can be detected using a number of methods, for example by monitoring production of cytokines or measuring uptake of tritiated thymidine. In the most preferred embodiment, interferon gamma production is monitored using Elispot assays.

The biological properties of the modular antibody according to the invention may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic with the appropriate half-life, effector function, apoptotic activity, cytotoxic or cytolytic activity. Any organism, preferably mammals, may be used for testing. For example because of their genetic similarity to humans, primates, monkeys can be suitable therapeutic models, and thus may be used to test the efficacy, toxicity, pharmacokinetics, pharmacodynamics, half-life, or other property of the modular antibody according to the invention. Tests of the substances in humans are ultimately required for approval as drugs, and thus of course these experiments are contemplated. Thus the modular antibodies of the present invention may be tested in humans to determine their therapeutic efficacy, toxicity, immunogenicity, pharmacokinetics, and/or other clinical properties. Especially those modular antibodies according to the invention that bind to single cell or a cellular complex through at least two binding motifs, preferably binding of at least three structures cross-linking target cells, would be considered effective in effector activity or preapoptotic or apoptotic activity upon cell targeting and cross-linking. Multivalent binding provides a relatively large association of binding partners, also called cross-linking, which is a prerequisite for apoptosis and cell death.

The modular antibody of the present invention may find use in a wide range of antibody products. In one embodiment the modular antibody of the present invention is used for therapy or prophylaxis, e.g. as an active or passive immunotherapy, for preparative, industrial or analytic use, as a diagnostic, an industrial compound or a research reagent, preferably a therapeutic. The modular antibody may find use in an antibody composition that is monoclonal or polyclonal. In a preferred embodiment, the modular antibodies of the present invention are used to capture or kill target cells that bear the target antigen, for example cancer cells. In an alternate embodiment, the modular antibodies of the present invention are used to block, antagonize, or agonize the target antigen, for example by antagonizing a cytokine or cytokine receptor.

In an alternately preferred embodiment, the modular antibodies of the present invention are used to block, antagonize, or agonize growth factors or growth factor receptors and thereby mediate killing the target cells that bear or need the target antigen.

In an alternately preferred embodiment, the modular antibodies of the present invention are used to block, antagonize, or agonize enzymes and substrate of enzymes.

In a preferred embodiment, a modular antibody is administered to a patient to treat a specific disorder. A “patient” for the purposes of the present invention includes both humans and other animals, preferably mammals and most preferably humans. By “specific disorder” herein is meant a disorder that may be ameliorated by the administration of a pharmaceutical composition comprising a modified immunoglobulin of the present invention.

In one embodiment, a modular antibody according to the present invention is the only therapeutically active agent administered to a patient. Alternatively, the modular antibody according the present invention is administered in combination with one or more other therapeutic agents, including but not limited to cytotoxic agents, chemotherapeutic agents, cytokines, growth inhibitory agents, anti-hormonal agents, kinase inhibitors, anti-angiogenic agents, cardioprotectants, or other therapeutic agents. The modular antibody may be administered concomitantly with one or more other therapeutic regimens. For example, a modular antibody of the present invention may be administered to the patient along with chemotherapy, radiation therapy, or both chemotherapy and radiation therapy. In one embodiment, the modular antibody of the present invention may be administered in conjunction with one or more antibodies, which may or may not comprise a modular antibody of the present invention. In accordance with another embodiment of the invention, the modular antibody of the present invention and one or more other anti-cancer therapies is employed to treat cancer cells ex vivo. It is contemplated that such ex vivo treatment may be useful in bone marrow transplantation and particularly, autologous bone marrow transplantation. It is of course contemplated that the antibodies of the invention can be employed in combination with still other therapeutic techniques such as surgery.

A variety of other therapeutic agents may find use for administration with the modular antibody of the present invention. In one embodiment, the modular antibody is administered with an anti-angiogenic agent, which is a compound that blocks, or interferes to some degree, the development of blood vessels. The anti-angiogenic factor may, for instance, be a small molecule or a protein, for example an antibody, Fc fusion molecule, or cytokine, that binds to a growth factor or growth factor receptor involved in promoting angiogenesis. The preferred anti-angiogenic factor herein is an antibody that binds to Vascular Endothelial Growth Factor (VEGF). In an alternate embodiment, the modular antibody is administered with a therapeutic agent that induces or enhances adaptive immune response, for example an antibody that targets CTLA-4. In an alternate embodiment, the modified immunoglobulin is administered with a tyrosine kinase inhibitor, which is a molecule that inhibits to some extent tyrosine kinase activity of a tyrosine kinase. In an alternate embodiment, the modular antibody of the present invention are administered with a cytokine. By “cytokine” as used herein is meant a generic term for proteins released by one cell population that act on another cell as intercellular mediators including chemokines.

Pharmaceutical compositions are contemplated wherein modular antibodies of the present invention and one or more therapeutically active agents are formulated. Stable formulations of the modular antibodies of the present invention are prepared for storage by mixing said immunoglobulin having the desired degree of purity with optional pharmaceutically acceptable carriers, excipients or stabilizers, in the form of lyophilized formulations or aqueous solutions. The formulations to be used for in vivo administration are preferably sterile. This is readily accomplished by filtration through sterile filtration membranes or other methods. The modular antibody and other therapeutically active agents disclosed herein may also be formulated as immunoliposomes, and/or entrapped in microcapsules.

Administration of the pharmaceutical composition comprising a modular antibody of the present invention, preferably in the form of a sterile aqueous solution, may be done in a variety of ways, including, but not limited to, orally, subcutaneously, intravenously, intranasally, intraotically, transdermally, mucosal, topically (e.g., gels, salves, lotions, creams, etc.), intraperitoneally, intramuscularly, intrapulmonary (e.g., AERx™ inhalable technology commercially available from Aradigm, or Inhance™ pulmonary delivery system commercially available from Inhale Therapeutics), vaginally, parenterally, rectally, or intraocularly.

The foregoing description will be more fully understood with reference to the following examples. Such examples are, however, merely representative of methods of practicing one or more embodiments of the present invention and should not be read as limiting the scope of invention.

### EXAMPLES

### Example 1

### C-Terminal Disulfide Bridge in Fc

In order to increase the stability of a homodimeric Fc fragment, an interchain disulfide bridge was engineered at the C-terminus of the CH3 domain.

Mutating residues in the CH3 domain C-terminally to Ser124 (IMGT numbering) structurally allows the formation of a disulfide bridge, to construct a homodimeric Fc fragment with a C-terminal disulfide bond. According to this example, the residues that were introduced as mutations in the CH3 domain were the three C-terminal residues of the CL domain, GlyGluCys. The mutations that were introduced in the CH3 domain were therefore: Pro125Gly, Gly129Glu, Lys130Cys (IMGT numbering).

Sequence and Translation of the Mutated Fc:

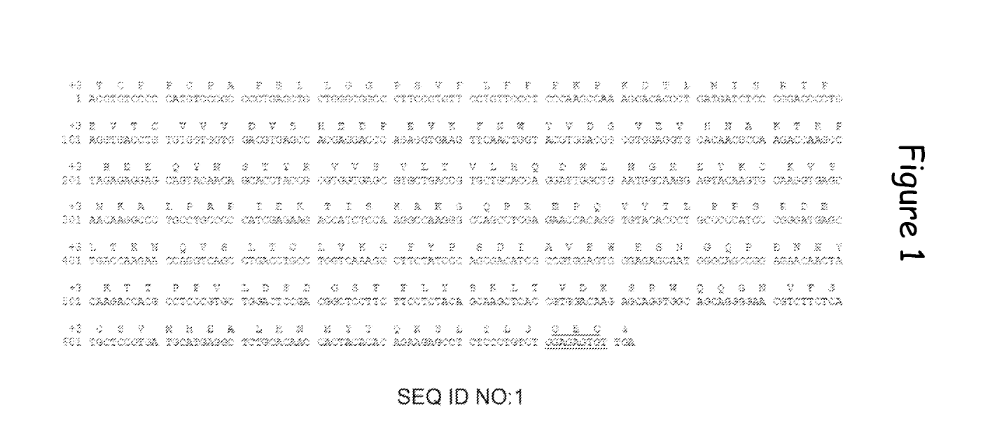

The sequence of the mutant Fc is provided in FIG. 1 (SEQ ID No. 1—nucleotide sequence; SEQ ID No. 2—protein sequence). The mutation was introduced using standard methods for site directed mutagenesis. In particular, the Quikchange kit (Stratagene) was used. The mutagenic primer CH3SSSNot had the following sequence:

CH3SSSNot (47 bp)
SEQ ID No. 3
5′-attcgcggcc gctcaacact ctccagacag ggagaggctc
ttctgtg

Before expression, the sequence of the mutated Fc was verified by DNA sequencing.

Genes encoding Fc and Fc with C-terminal cystein were cloned into Pichia pastoris expression vector pPICZalphaA between EcoRI and NotI sites in frame with the Saccharomyces cerevisiae alpha-factor leader sequence for secretion to the supernatant. After linearization with SacI the plasmids were transformed into Pichia pastoris X33 using electroporation and transformants were selected on YPD medium with 250 μg/ml zeocin. P. pastoris colonies were inoculated into YPG medium and production of the recombinant protein was induced using YP with 1% methanol. Induction was continued for three days according to standard protocols (Invitrogen).

Supernatant was harvested by centrifugation at 3000 rpm, 15 min, 4° C., and cleared with another centrifugation at 8000 rpm, 15 min, at 4° C. It was then loaded on Protein A HP column, previously equilibrated with 0.1M Na-phosphate buffer, pH=7.0. After loading, the column was washed with the same buffer and protein was eluted with 0.1M glycine, pH=3.5, and neutralized immediately with 2M Tris-base. Fractions containing protein were pooled and dialysed against 100× volume of 1×PBS, pH=7.2, at 4° C.

Differential Scanning Calorimetry (DSC) was used to assess the thermostability of the proteins. DSC measurements were performed in a Microcal VP-DSC instrument with a heating rate of 60° C./h. Protein concentration was 0.25 mg/ml. Firstly, the thermal scan was made from 20 to 100° C. With a fresh protein sample, an annealed scan consisting of the following 3 steps was made:

1. 20-72° C., followed by cooling to 20° C.

2. 20-100° C., followed by cooling to 20° C.

3. 20-100° C.

The first unfolding event was completely reversible when heating did not exceed 72° C. Heating to 100° C. produced an irreversible thermal unfolding as revealed by the third scan. Therefore, for evaluation of the thermal stability of the protein, the initial scan was used, and the signal given by third scan was used as a baseline. Tm (melting points) were read as mid-transition points. Enthalpies were calculated with Microcal Origin for DSC using a non-2-state-model with 3 peaks.

TABLE 4
Melting points (Tm) as determined by DSC

| | Tm1 | | Tm2 | | Tm3 | |
|---|---|---|---|---|---|---|
| Fcwt | 66.62 | ±0.012 | 77.50 | ±0.029 | 82.60 | ±0.013 |
| Fcwt_ss | 66.51 | ±0.0094 | 83.74 | ±0.014 | 91.65 | ±0.0064 |

TABLE 5
Enthalpies

| | Transition | | | | | |
|---|---|---|---|---|---|---|
| | 1^{st} | | 2^{nd} | | 3^{rd} | |
| | ΔH1 | ΔHν1 | ΔH2 | ΔHν2 | ΔH3 | ΔHν3 |
| Fcwt | 1.074E5 | 1.067E5 | 1.138E5 | 1.224E5 | 6.616E4 | 2.182E5 |
| Fcwt_ss | 1.223E5 | 9.199E4 | 8.073E4 | 1.198E5 | 7.625E4 | 2.233E5 |

The large positive shift, 6,24° C. and 9,05° C., in the melting points of thermal denaturation, Tm2 and Tm3, respectively, signifies an increased thermal stability of the mutant in respect to the wild-type Fc.

The mutant Fc is used as a scaffold to provide a library of Fc variants with randomized sequences in the structural loop region to select members of the library with new antigen binding sites.

### Example 2

### Intradomain Disulfide Bridges in Fc Wild-Type

In order to increase the stability of a homodimeric Fc fragment, two different intrachain disulfide bridges were engineered in the CH3 domain.

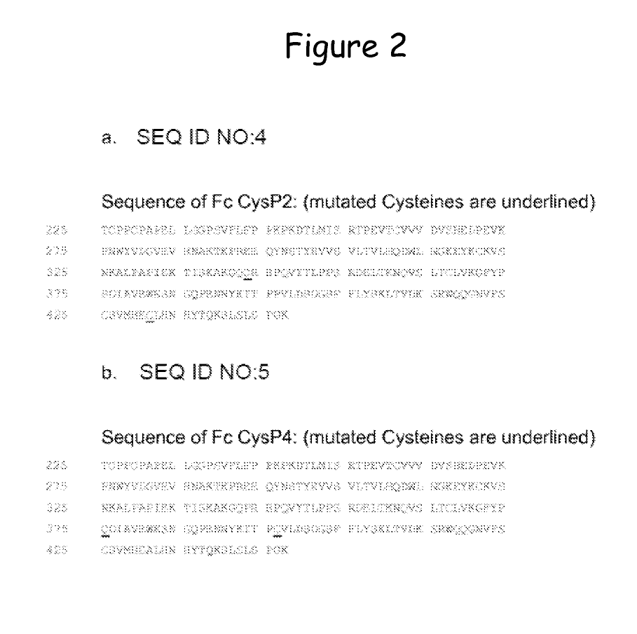

By mutating Pro343Cys and Ala431Cys, Fc wt CysP2 was generated (all numberings according to the Kabat numbering scheme). The two residues that are mutated to Cys in this clone are located near the N-terminus of the CH3 domain (Pro343) and in the FG loop (Ala431) (IMGT numbers of CysP2: 1.2 and 110). The sequence is provided in FIG. 2 a. (mutated Cysteines are underlined) and SEQ ID No. 4.

By mutating Ser375Cys and Pro396Cys, Fc wt CysP4 was generated. The two residues that are mutated to Cys in this clone are located in the BC loop of the CH3 domain (Ser375) and in the D sheet (Pro396) (IMGT numbers of CysP4: 33 and 83). The sequence is provided in FIG. 2 b. (mutated Cysteines are underlined) and SEQ ID No. 5.

The mutations were introduced into the DNA sequence coding for Fc wild-type using standard methods for site directed mutagenesis. In particular, the Quikchange kit (Stratagene) was used. Before expression, the sequence of the mutated Fc was verified by DNA sequencing.

Cloning, expression, purification and DSC measurements of Fc wild-type, Fc CysP2 and Fc CysP4 were performed as described in example 1. Results of the DSC measurements, showing increased thermostability of the CH3 domain in clones Fc CysP2 and Fc CysP4 are shown in Table 6.

TABLE 6
Results of DSC measurements

| | Tₘ1 | ΔH1 | ΔHᵥ1 | Tₘ2 | ΔH2 | ΔHᵥ2 | Tₘ3 | ΔH3 | ΔHᵥ3 |
|---|---|---|---|---|---|---|---|---|---|
| Construct | (° C.) | (kcal/mol) | (kcal/mol) | (° C.) | (kcal/mol) | (kcal/mol) | (° C.) | (kcal/mol) | (kcal/mol) |
| Fc wt | 65.9 | 129.5 | 90.7 | 78.1 | 72.6 | 136.7 | 82.6 | 59.7 | 234.1 |
| Fc CysP2 | 64.0 | 88.5 | 108.8 | 86.6 | 67.9 | 129.5 | 92.8 | 109.6 | 226.3 |
| Fc CysP4 | 64.1 | 119.3 | 100.9 | 82.9 | 106.9 | 124.4 | 87.3 | 65.3 | 230.1 |

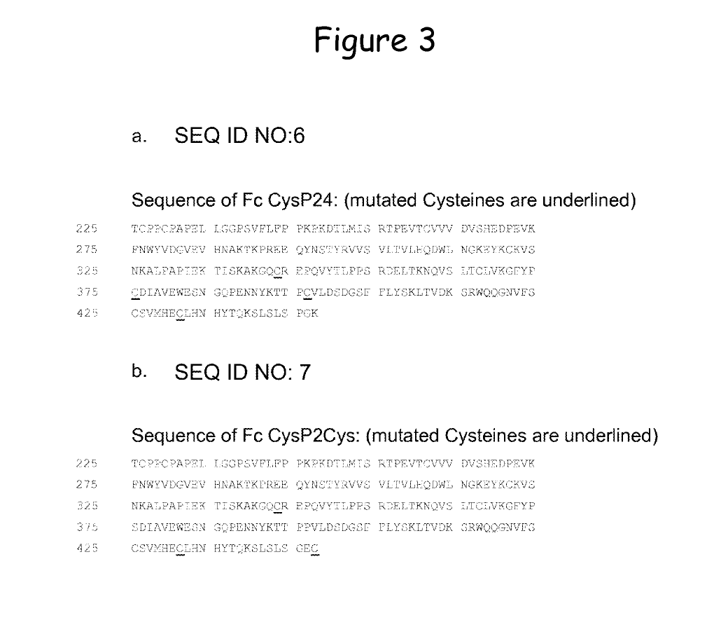

Furthermore, combinations of disulfide bridges were made:
Disulfide bridge CysP2 was combined in a single clone with disulfide bridge CysP4, designated CysP24. The sequence is provided in FIG. 3 a. (mutated Cysteines are underlined) and SEQ ID No. 6.
Disulfide bridge CysP2 was combined in a single clone with the C-terminal disulfide bridge from Example 1, designated CysP2Cys. The sequence is provided in FIG. 3 b. (mutated Cysteines are underlined) and SEQ ID No. 7.

Cloning, expression, purification and DSC measurements were performed as described above. Results of the DSC measurements, showing increased thermostability of the CH3 domain (Tₘ2 and Tₘ3) in clones Fc CysP24 and Fc CysP2Cys are shown in Table 7.

TABLE 7
Results of DSC measurements

| | Tₘ1 | ΔH1 | ΔHᵥ1 | Tₘ2 | ΔH2 | ΔHᵥ2 | Tₘ3 | ΔH3 | ΔHᵥ3 |
|---|---|---|---|---|---|---|---|---|---|
| Construct | (° C.) | (kcal/mol) | (kcal/mol) | (° C.) | (kcal/mol) | (kcal/mol) | (° C.) | (kcal/mol) | (kcal/mol) |
| Fc wt | 65.9 | 129.5 | 90.7 | 78.1 | 72.6 | 136.7 | 82.6 | 59.7 | 234.1 |
| Fc wt CysP24 | 61.0 | 112.0 | 88.6 | 92.8 | 150.2 | 76.5 | 97.8 | 84.2 | 230.1 |
| Fc wt CysP2Cys | 63.6 | 76.2 | 94.0 | 96.6 | 105.5 | 108.3 | 101.1 | 79.9 | 233.9 |

### Example 3

### Intra- and Interdomain Disulfide Bridges in Fc H10-03-6

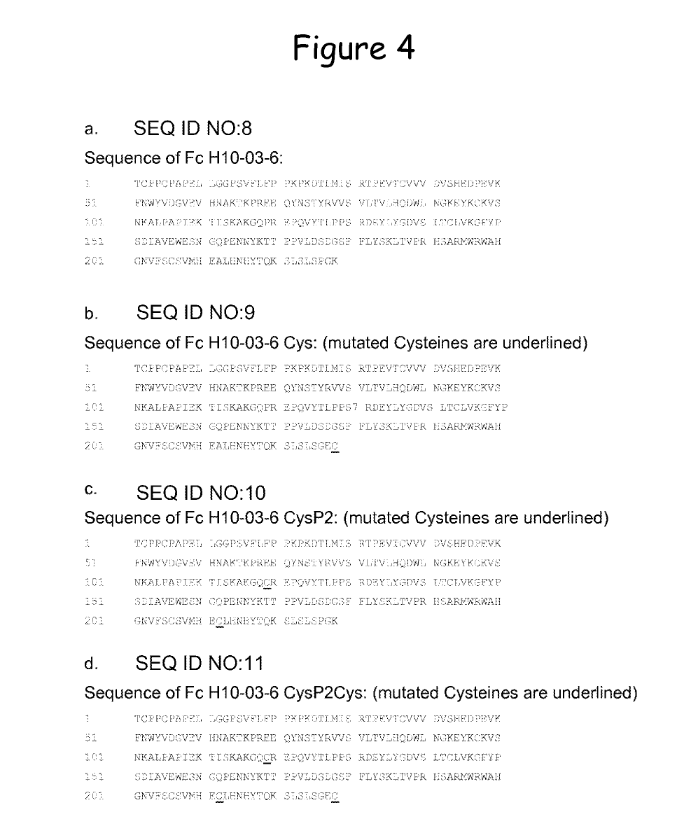

Previously, an Fc with mutations in structural loops of the CH3 domains was generated which binds specifically to HER2/neu (according to WO2009/000006A1). The sequence of Fc H10-03-6 is provided in FIG. 4 a. and SEQ ID No. 8. It was found that the thermostability of this clone is decreased relative to Fc wild-type. Therefore, attempts to stabilise it by introduction of disulfide bridges were undertaken.

Into this HER2/neu specific Fc, the C-terminal disulfide bridge according to Example 1 was introduced to generate clone H10-03-6 Cys (the sequence is provided in FIG. 4 b. (mutated Cysteines are underlined) and SEQ ID No. 9). Furthermore, the disulfide bridge CysP2 according to Example 2 was introduced (H10-03-6 CysP2, the sequence is provided in FIG. 4 c. (mutated Cysteines are underlined) and SEQ ID No. 10) as well as a combination of these two disulfide bridges (H10-03-6 CysP2Cys, the sequence is provided in FIG. 4 d. (mutated Cysteines are underlined) and SEQ ID No. 11).

Cloning, expression, purification and DSC measurements were performed as described above. Results of the DSC measurements, showing increased thermostability of the CH2 (Tₘ1) and CH3 domains (Tₘ2) are shown in Table 8. It should be noted, that in all H10-03-6 clones, the third transition point of thermal denaturation which can be seen in Fc wild-type is not observed.

TABLE 8
Results of DSC measurements

| | | | | | ΔH2 | ΔHᵥ2 |
|---|---|---|---|---|---|---|
| | Tₘ1 | ΔH1 | ΔHᵥ1 | Tₘ2 | (kcal/ | (kcal/ |
| Construct | (° C.) | (kcal/mol) | (kcal/mol) | (° C.) | mol) | mol) |
| H10-03-6 | 61.1 | 112.8 | 113.6 | 65.2 | 88.3 | 204.2 |
| H10-03-6 Cys | 65.9 | 95.0 | 114.1 | 73.9 | 50.8 | 151.6 |
| H10-03-6 CysP2 | 62.7 | 25.0 | 106.8 | 77.0 | 20.1 | 137.4 |
| H10-03-6 CysP2cys | 63.4 | 92.4 | 99.7 | 85.1 | 72.6 | 128.6 |

### Example 4

### Intra- and Interdomain Disulfide Bridges in Fc EAM151-5

In another experiment, a new engineered Fc clone binding to antigen X was selected (according to WO2009/000006A1). This clone is designated EAM151-5. It was found that the thermostability of this clone is decreased relative to Fc wild-type. Therefore, attempts to stabilise it by introduction of disulfide bridges were undertaken by introducing the following disulfide bridges and combinations of disulfide bridges:
EAM151-5 Cys
EAM151-5 CysP2
EAM151-5 CysP2Cys
EAM151-5 CysP4Cys
EAM151-5 CysP24

Cloning, expression, purification and DSC measurements were performed as described above. Results of the DSC measurements, showing increased thermostability of the CH3 domains (Tₘ2 and Tₘ3) are shown in Table 9. It should be noted, that in EAM151-5 as well as in some of the stabilised variants, the third transition point of thermal denaturation which can be seen in Fc wild-type is not observed. However, clones EAM151-5 CysP2Cys and EAM151-5 CysP24 are stabilised to such an extent that the Tₘ3 can be observed.

TABLE 9
Results of DSC measurements

| | Tₘ1 | ΔH1 | ΔHᵥ1 | Tₘ2 | ΔH2 | ΔHᵥ2 | Tₘ3 | ΔH3 | ΔHᵥ3 |
|---|---|---|---|---|---|---|---|---|---|
| Construct | (° C.) | (kcal/mol) | (kcal/mol) | (° C.) | (kcal/mol) | (kcal/mol) | (° C.) | (kcal/mol) | (kcal/mol) |
| EAM151-5 | 69.1 | 105.1 | 190.7 | 71.0 | 96.0 | 319.0 | | | |
| EAM151-5 Cys | 68.0 | 189.4 | 100.4 | 73.3 | 11.4 | 315.6 | | | |
| EAM151-5 CysP2 | 66.2 | 242.1 | 77.0 | 75.3 | 57.8 | 181.9 | | | |
| EAM151-5 | 65.4 | 160.2 | 83.7 | 77.6 | 52.3 | 124.8 | 82.8 | 44.3 | 190.4 |
| CysP2Cys | | | | | | | | | |
| EAM151-5 | 66.4 | 200.9 | 91.2 | 77.7 | 50.6 | 174.9 | | | |
| CysP4Cys | | | | | | | | | |
| EAM151-5 CysP24 | 62.4 | 147.9 | 90.3 | 74.9 | 52.7 | 117.5 | 80.6 | 76.9 | 179.2 |

1. A multidomain modular antibody comprising at least one constant antibody domain, wherein a parent antibody molecule is mutated to form an artificial disulfide bridge in the multidomain modular antibody by introducing at least one Cys residue into the amino acid sequence of the parent antibody molecule through mutagenesis of said constant domain to obtain an intra-domain or inter-domain disulfide bridge within the framework region of the multidomain modular antibody.

2. The modular antibody of claim 1, wherein the modular antibody comprises at least two constant domains connected by said artificial disulfide bridge.

3. The modular antibody of claim 1, wherein the modular antibody comprises an antigen-binding region.

4. The modular antibody of claim 1, wherein the modular antibody is a full-length antibody or a part of a full-length antibody.

5. The modular antibody of claim 1, wherein said constant domain contributes to the antigen-binding function of the modular antibody.

6. The modular antibody of claim 1, wherein said at least one Cys residue is introduced at an amino acid position in the amino acid sequence of the parent antibody molecule which is not within an antigen binding site of the antibody.

7. (canceled)

8. A Library of modular antibodies according to claim 1, wherein said modular antibodies are mutagenized to obtain a randomized amino acid sequence within a loop region of each of the modular antibodies.

9. A method of producing a modular antibody, which comprises the steps of:
(a) providing a parent antibody molecule comprising at least two antibody domains, wherein at least one of the antibody domains is a constant domain,
(b) mutating said constant domain to introduce a Cys residue within the framework region of said constant domain, and
(c) expressing said modular antibody at oxidizing conditions to form a new disulfide bridge within the molecule.

10. The method of claim 9, wherein at least two constant domains are mutated to introduce a Cys residue.

11. The method of claim 9, wherein said constant domain contributes to antigen-binding.

12. The method of claim 9, wherein said Cys residue is introduced at an amino acid position in the amino acid sequence of the modular antibody which is not within an antigen binding site of the antibody.

13. The method of claim 9, wherein said modular antibody is expressed by a host cell at disulfide forming conditions.

14. The method of claim 9, wherein the modular antibody expressed in step (c) exhibits increased thermostability when compared to the parent antibody molecule.

15. The method of claim 9, wherein the modular antibody expressed in step (c) exhibits improved antigen-binding when compared to the parent antibody molecule.

16. The modular antibody of claim 4, wherein the modular antibody is selected from the group consisting of a Fab molecule, an Fc domain, and a molecule comprising a combination of at least one constant domain with at least one other domain selected from the group consisting of a constant domain and a variable domain.


SEQUENCE LISTING (11 sequences)

SEQ ID NO: 1 - DNA, 672 bp, Artificial Sequence
  mutant Fc - nucleotide sequence
acgtgtcccccatgtcccgcccctgagctgctgggcggcccttccgtgttcctgttccctcccaagccaaaggacaccctgatgatctcccggacccctgaggtgacctgtgtggtggtggacgtgagccacgaggacccagaggtgaagttcaactggtacgtggacggcgtggaggtgcacaacgccaagaccaagcctagagaggagcagtacaacagcacctaccgcgtggtgagcgtgctgaccgtgctgcaccaggattggctgaatggcaaggagtacaagtgcaaggtgagcaacaaggccctgcctgcccccatcgagaagaccatctccaaggccaagggccagcctcgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctggagagtgttga

SEQ ID NO: 2 - protein, 223 aa, Artificial Sequence
  mutated Fc - protein sequence
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 3 - DNA, 47 bp, Artificial Sequence
  Primer CH3SSSNot
attcgcggccgctcaacactctccagacagggagaggctcttctgtg

SEQ ID NO: 4 - protein, 223 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQCREPQVYTLPPSRDELTKNQVSLTCLVKGFYPSDIAVEWESNGQPENNYKTTPPVLDSDGSFFLYSKLTVDKSRWQQGNVFSCSVMHECLHNHYTQKSLSLSPGK

SEQ ID NO: 5 - protein, 223 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPCDIAVEWESNGQPENNYKTTPCVLDSDGSFFLYSKLTVDKSRWQQGNVFSCSVMHEALHNHYTQKSLSLSPGK

SEQ ID NO: 6 - protein, 223 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQCREPQVYTLPPSRDELTKNQVSLTCLVKGFYPCDIAVEWESNGQPENNYKTTPCVLDSDGSFFLYSKLTVDKSRWQQGNVFSCSVMHECLHNHYTQKSLSLSPGK

SEQ ID NO: 7 - protein, 223 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQCREPQVYTLPPSRDELTKNQVSLTCLVKGFYPSDIAVEWESNGQPENNYKTTPPVLDSDGSFFLYSKLTVDKSRWQQGNVFSCSVMHECLHNHYTQKSLSLSGEC

SEQ ID NO: 8 - protein, 228 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLYGDVSLTCLVKGFYPSDIAVEWESNGQPENNYKTTPPVLDSDGSFFLYSKLTVPRHSARMWRWAHGNVFSCSVMHEALHNHYTQKSLSLSPGK

SEQ ID NO: 9 - protein, 228 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLYGDVSLTCLVKGFYPSDIAVEWESNGQPENNYKTTPPVLDSDGSFFLYSKLTVPRHSARMWRWAHGNVFSCSVMHEALHNHYTQKSLSLSGEC

SEQ ID NO: 10 - protein, 228 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQCREPQVYTLPPSRDEYLYGDVSLTCLVKGFYPSDIAVEWESNGQPENNYKTTPPVLDSDGSFFLYSKLTVPRHSARMWRWAHGNVFSCSVMHECLHNHYTQKSLSLSPGK

SEQ ID NO: 11 - protein, 228 aa, Artificial Sequence
  mutated human Fc
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQCREPQVYTLPPSRDEYLYGDVSLTCLVKGFYPSDIAVEWESNGQPENNYKTTPPVLDSDGSFFLYSKLTVPRHSARMWRWAHGNVFSCSVMHECLHNHYTQKSLSLSGEC